(19) United States
(12) Patent Application Publication
(10) Pub. No.: US 2006/0171945 A1
(43) Pub. Date: Aug. 3, 2006

(54) Ip receptor antagonists for the treatment of pathological uterine conditions

(76) Inventors: Hilary Octavia Dawn Critchley, Edinburg (GB); Henry Nicolas Jabbour, Edinburgh (GB)

Correspondence Address: Michael L Goldman;Nixon Peabody, Clinton Square, P O Box 31051, Rochester, NY, 14603-1051, US

(21) Appl. No.: 10/545,478

(22) PCT Filed: Feb. 16, 2004
(86) PCT No.: PCT/GB04/00588

§ 371 Date: Aug. 15, 2005

(30) Foreign Application Priority Data
Feb. 14, 2003 (GB) 0303430.9
Jul. 1, 2003 (GB) 0315322.8

(51) Int. Cl. A61K 39/395; A61K 38/10; A61K 38/08; A61K 31/5377; A61K 31/4196; A61K 31/557

(52) U.S. Cl. 424/145.1; 514/401; 514/235.5; 514/383; 514/573; 514/16; 514/17; 514/13; 514/14; 514/15; 514/456

(57) ABSTRACT

A method of combating a pathological condition of the uterus in a female individual, the method comprising administering to the individual at least one agent that is an antagonist of the IP receptor and/or a PGIS inhibitor. The pathological condition of the uterus is uterine carcinoma, menorrhagia, dysmenorrhoea or an endomenstrual myometrial pathological condition.

The present invention relates to methods of treatment, and in particular methods of treating uterine pathological conditions.

Pathological conditions of the uterus represent a serious health problem in women, particularly women of the western world. Such pathological conditions include uterine cancers, endometrial or myometrial pathological conditions such as endometriosis (endometrial) and fibroids (myometrial), menorrhagia and dysmenorrhoea.

Epithelial cells of the human endometrium are highly vulnerable to neoplastic transformation. In the western world, endometrial carcinoma is the most common gynaecologic malignancy. Endometrial cancer can arise from several cell types but the glandular epithelium is the most common progenitor (adenocarcinomas account for 80-90% of uterine tumours). Endometrial cancer is predominantly a post-menopausal disease where incidence is uncommon below the age of forty and peaks by about seventy years of age. The incidence of endometrial cancer has been increasing steadily in the western world during the last fifty years and this has been attributed largely to increased life expectancy and improved detection methods (Gordon & Ireland, 1994; Mant & Vessey, 1994).

Endometriosis is the ectopic implantation and growth of endometrium and can therefore be considered as abnormal growth of cells of the endometrium. Adenomyosis is a form of endometriosis where the ectopic endometrium is implanted in the myometrium.

Menorrhagia is over-abundance of the menstrual discharge. Dysmenorrhoea means painful menstruation. Menorrhagia and dysmenorrhoea affect many women, particularly in the western world, and represent a significant health problem. At least one in 20 women in the UK aged between 34 and 49 years will consult their general practitioners because of menstrual problems. These women account for more than one in ten of all gynaecological referrals and cost the NHS in excess of £7 million per year for medical prescriptions alone.

Perceived abnormal vaginal bleeding is said to account for 70% of the at least 70,000 hysterectomies performed each year. At present, the treatments used for menorrhagia include tranexamic acid or mefenamic acid. In severe cases the treatment is hysterectomy (vaginal or abdominal) but this is a major operation with serious morbidity and some risk of death. A review of treatments for menorrhagia is Stirrat (1999) The Lancet 353, 2175-2176. The development of further and alternative therapies is desirable.

Cyclooxygenase (COX) enzymes, also called prostaglandin endoperoxide synthase (PGHS), catalyse the rate limiting step in the conversion of arachidonic acid to prostaglandin H₂ (PGH₂). In turn PGH₂ serves as a substrate for specific prostaglandin synthase enzymes that synthesise the natural prostaglandins. These are named according to the prostaglandin they produce such that prostaglandin D₂ is synthesised by prostaglandin-D-synthase, prostaglandin E₂ (PGE₂) by prostaglandin-E-synthase (PGES), prostaglandin F_{2α} (PGF_{2α}) by prostaglandin-F-synthase (PGFS), and prostacyclin (prostaglandin I₂, PGI₂) by prostaglandin-I-synthase (PGIS).

To date, there are two identified isoforms of the COX enzyme, COX-1 and COX-2 (DeWitt, 1991). COX-1 is constitutively expressed in many tissues and cell types and generates prostaglandins for normal physiological function (Herschman, 1996). By contrast, the expression of COX-2 is rapidly induced following stimulation of quiescent cells by growth factors, oncogenes, carcinogens and tumour-promoting phorbol esters (Herschman, 1996; Subbaramaiah et al, 1996).

Prostacyclin (PGI₂) has been characterised as a vasodilator and a potent inhibitor of platelet aggregation (Smyth & Fitzgerald, 2002) and as such plays an essential role in the maintenance of vascular haemostasis. It is the main prostanoid synthesised by vascular endothelium and acts as a smooth muscle relaxant. PGI₂ elicits its effects on target cells by interaction with its G protein-coupled receptor (IP), which has a typical seven-transmembrane structure (Narumiya et al, 1999). The IP receptor can stimulate both Gs and Gq species of G proteins causing an increase in cAMP generation and in phosphatidylinositol response.

PGI₂ has been implicated in the aetiology of menorrhagia, which is a major problem in women's reproductive health, accounting for 11% of all gynaecological referrals and incurring drug costs amounting to £7 m in 1995 (Cooper et al, 2001). There is evidence for increased synthesis of PGI₂ (Smith et al, 1981) or an increase in PGI₂ concentration relative to thromboxane A₂ (Makarainen & Ylikorkala 1986) in women with menorrhagia compared with controls. However, little information is available on the temporal or spatial expression of PGIS or IP in the endometrium across the menstrual cycle.

The pharmacology, molecular biology and signal transduction of the platelet prostacyclin receptor are reviewed by Armstrong (1996, Pharmacol. Ther. 72(3), p171-191).

The cloning and sequence of the human IP receptor has been reported in EP 0 753 528 A1 (Ono Pharmaceutical Co.); U.S. Pat. Nos. 5,728,808 and 6,365,360 (assigned to Merck Frosst Canada & Co.); Boie et al “Cloning and expression of a cDNA for the human prostanoid IP receptor” J. Biol. Chem. 269 (16), 12173-12178 (1994); Katsuyama et al, “Cloning and expression of a cDNA for the human prostacyclin receptor” FEBS Lett. 344 (1), 74-78 (1994);

Nakagawa et al, “Molecular cloning of human prostacyclin receptor cDNA and its gene expression in the cardiovascular system” Circulation 90 (4), 1643-1647 (1994); Duncan et al, “Chromosomal localization of the human prostanoid receptor gene family” Genomics 25 (3), 740-742 (1995); and Ogawa et al, “Structural organization and chromosomal assignment of the human prostacyclin receptor gene” Genomics 27 (1), 142-148 (1995), all of which are incorporated herein by reference.

The cDNA sequence of the human IP receptor is given in Genbank Accession No. NM_{—}000960, and the amino acid sequence given in Genbank Accession No. NP_{—}000951. Some sequence variation has been identified within the human IP receptor gene, as is noted in Genbank Accession No. NM_{—}000960.

The cloning and sequence of human PGIS has been reported in Miyata et al, “Molecular cloning and expression of human prostacyclin synthase” Biochem. Biophys. Res. Commun. 200 (3): 1728-1734 (1994); Nakayama et al, “Organization of the human prostacyclin synthase gene” Biochem. Biophys. Res. Commun. 221 (3): 803-806 (1996); Yokoyama et al, “Human gene encoding prostacyclin synthase (PTGIS): genomic organization, chromosomal localization, and promoter activity” Genomics 36 (2): 296-304 (1996); Wang et al, “Organization of the gene encoding human prostacyclin synthase” Biochem. Biophys. Res. Commun. 226 (3): 631-637 (1996); and Chevalier et al, “Characterization of new mutations in the coding sequence and 5′-untranslated region of the human prostacylcin synthase gene (CYP8A1)” Hum. Genet. 108 (2): 148-155 (2001).

The cDNA sequence of human PGIS receptor is given in Genbank Accession No. NM_{—}000961, and the amino acid sequence given in Genbank Accession No. NP_{—}000952. Some sequence variation has been identified within the human PGIS gene, as is noted in Genbank Accession No. NM_{—}000961.

We have now shown that both PGIS and IP are up-regulated in the early proliferative phase of the menstrual cycle, with increased signalling of the IP receptor via the cAMP pathway during this phase.

These observations have led the inventors to the surprising and unexpected belief that antagonising the IP receptor can combat pathological conditions of the uterus.

Antagonists of the IP receptor have been suggested for use in treatment of a range of bladder disorders, to prevent conditions associated with excessive bleeding such as haemophilia and haemorrhaging, to relieve hypotension related to septic shock, to reduce oedema formation, and to be useful in respiratory allergies and respiratory conditions such as asthma, inflammation, and in pain conditions such as inflammatory pain and premenstrual pain (WO 02/40453; WO 02/070514; WO 02/070500; WO 01/68591, EP 0 902 018; U.S. Pat. No. 6,184,242) for cardiovascular disease (WO 79/00744) for ameliorating neuropathic disorders (WO 01/10433) and treating neurodegenerative diseases (WO 01/10455).

They have not, however, been previously suggested to be useful in combating uterine pathological conditions, such as uterine cancers, endometriosis, fibroids, menorrhagia and dysmenorrhoea.

Current treatment of uterine pathologies includes the use of COX-inhibitors.

While COX-inhibitors have shown some therapeutic potential, they prevent the synthesis of a number of prostaglandins, of which only some are harmful, and some have beneficial effects. There is thus a need in the art for methods for treating uterine pathologies by specifically inhibiting the action of specified prostaglandins.

A first aspect of the invention provides a method combating a pathological condition of the uterus in a female individual, the method comprising administering to the individual at least one agent that is an antagonist of the IP receptor and/or an inhibitor of PGIS.

The IP receptor is the receptor that binds prostaglandin I₂ (PGI₂).

The pathological conditions of the uterus treatable by the methods of the invention include, but are not limited to, any pathological condition wherein IP receptors are upregulated in proliferating tissue.

Typically, the pathological condition of the uterus is any one of uterine cancer such as uterine carcinoma, menorrhagia, dysmenorrhoea, an endometrial pathological condition such as endometriosis including adenomyosis, or a myometrial pathological condition such as fibroids (leiomyomas) or leiomyosarcomas which are fibroids which have become malignant. Thus, typically, the uterine pathological condition is one which is associated with abnormal growth of cells of the myometrium or endometrium.

Certain uterine pathological conditions are believed to be associated with overproliferation of the epithelium.

It is believed that premenstrual pain is not a pathological condition of the uterus.

Premenstrual pain is typically experienced in the days preceding menstruation, which is distinguished from the uterine pathological condition dysmenorrhoea that occurs during menstruation. However, for the avoidance of doubt, the term “pathological condition of the uterus” or “uterine pathological condition” as used herein does not include premenstrual pain.

In one particular embodiment, the invention includes a method of combating a pathological condition of the uterus other than menorrhagia in a female individual, the method comprising administering to the individual at least one agent that is an antagonist of the IP receptor.

By combating a pathological condition of the uterus we include alleviating symptoms of the condition (palliative use), or treating the condition, or preventing the condition (prophylactic use).

The invention thus includes the treatment of any of a uterine cancer such as carcinoma, menorrhagia, dysmenorrhoea, an endometrial pathological condition such as endometriosis, and a myometrial pathological condition such as fibroids, with at least one agent that is an antagonist of the IP receptor.

The invention also includes alleviating symptoms of any of a uterine cancer such as carcinoma, menorrhagia, dysmenorrhoea, an endometrial pathological condition such as endometriosis, and a myometrial pathological condition such as fibroids, with at least one agent that is an antagonist of the IP receptor.

The invention further includes preventing, or preventing the symptoms of, any of a uterine cancer such as carcinoma, menorrhagia, dysmenorrhoea, an endometrial pathological condition such as endometriosis, and a myometrial pathological condition such as fibroids, with at least one agent that is an antagonist of the IP receptor.

The invention thus includes combating a uterine cancer, such as endometrial carcinoma, with at least one agent that is an antagonist of the IP receptor.

The invention also includes combating menorrhagia with at least one agent that is an antagonist of the IP receptor.

The invention further includes combating dysmenorrhoea with at least one agent that is an antagonist of the IP receptor.

The invention also includes combating an endometrial pathological condition, such as endometriosis, with at least one agent that is an antagonist of the IP receptor.

The invention still further includes combating a myometrial pathological condition, such as fibroids, with at least one agent that is an antagonist of the IP receptor.

It is possible for an individual to have more than one uterine pathological condition, and it is possible to use the methods of the present invention to treat the conditions together. For example, women often have menorrhagia and dysmenorrhoea together and the method may be used to treat both conditions in the same patient.

The patient may be any patient who is suffering from or who is at risk from a uterine pathological condition. Any premenopausal or perimenopausal woman is at risk of menorrhagia and/or dysmenorrhoea; however, menorrhagia is more common at the beginning and end of a woman's reproductive life so typically there is a greater risk when a woman's periods first start and in women over 40 years of age.

The patient to be treated may be any female individual who would benefit from such treatment. Typically and preferably the patient to be treated is a human female. However, the methods of the invention may be used to treat female mammals, such as the females of the following species: cows, horses, pigs, sheep, cats and dogs. Thus, the methods have uses in both human and veterinary medicine.

Typically, the agent that is an antagonist of the IP receptor is one which prevents or disrupts PGI₂-mediated signalling of the IP receptor, and which is suitable to be administered to a patient.

Preferably, an agent that is an antagonist of the IP receptor prevents or reduces the binding of PGI₂ to the IP receptor. Alternatively or additionally, the antagonist may affect the interaction between PGI₂ and the IP receptor, or the interaction between the IP receptor and the associated Gs or Gq protein, thus inhibiting or disrupting a PGI₂-IP mediated signal transduction pathway.

IP receptor antagonists are typically molecules which bind to the IP receptor, compete with the binding of the natural ligand PGI₂, and inhibit or disrupt the PGI₂-IP mediated signal transduction pathway.

In a preferred embodiment, preventing PGI₂ having its effect on the IP receptor includes occupying the PGI₂ binding site on the IP receptor, such that the natural ligand (PGI₂) is prevented from binding in a mode that would result in its normal mode of signalling.

Alternatively, the receptor antagonist may be a non-competitive IP receptor antagonist, for example a molecule which binds to the IP receptor without preventing PGI₂ binding thereto, but which disrupts the interaction between PGI₂ and the IP receptor, thus inhibiting or disrupting PGI₂-IP mediated signal transduction pathway.

Further alternatively, the non-competitive IP receptor antagonist may be a molecule which binds to the IP receptor and which disrupts the interaction between the IP receptor and the associated G protein, thus inhibiting or disrupting IP mediated signal transduction pathway.

In an alternative preferred embodiment, the agent may be an antagonist of PGI₂. PGI₂ antagonists are typically molecules which bind to PGI₂ and prevent or reduce PGI₂ binding to its receptor, which inhibits or disrupts the PGI₂-IP mediated signal transduction pathway. This is sometimes termed the ‘soluble receptor’ approach in which typically a part of the receptor binds to PGI₂.

The receptor antagonists are typically selective to the particular receptor and preferably have an equal or higher binding affinity to the IP receptor than does PGI₂. Although antagonists with a higher affinity for the receptor than the natural ligand are preferred, antagonists with a lower affinity may also be used, but it may be necessary to use these at higher concentrations.

Preferably, the IP receptor antagonists bind reversibly to the IP receptor.

Preferably, the IP receptor antagonists are selective for the IP receptor. Thus, typically, an IP receptor antagonist binds the IP receptor with a higher affinity than for any other prostaglandin receptor. Preferably, an IP receptor antagonist has at least a two-fold higher binding affinity for the IP receptor than for any other prostaglandin receptor. More preferably, an IP receptor antagonist has at least a three-fold, or at least a four-fold, or at least a five-fold, or at least a six-fold, or at least a seven-fold, or at least an eight-fold, or at least a nine-fold, or at least a ten-fold, or at least a fifty-fold, or at least a 100-fold, or at least a 500-fold, or at least a 1,000-fold higher binding affinity for the IP receptor than for any other prostaglandin receptor. Preferably, an IP receptor antagonist binds the IP receptor but does not substantially bind any other prostaglandin receptor.

EP 0 902 018 A2 (F. Hoffman-La Roche AG) discloses 2-(Arylphenyl)amino-imidazoline derivatives which are IP receptor antagonists. All of the disclosure in EP 0 902 018 A2 relating to IP receptor antagonists, is hereby incorporated herein by reference.

U.S. Pat. No. 6,184,242 (assigned to Syntex USA) discloses 2-(substituted-phenyl)amino-imidazoline derivatives which are IP receptor antagonists. All of the disclosure in U.S. Pat. No. 6,184,242 relating to IP receptor antagonists, is hereby incorporated herein by reference.

WO 02/070514 (F. Hoffman-La Roche AG) discloses alkoxycarbonylamino heteroaryl carboxylic acid derivatives which are IP receptor antagonists. Of these, the compounds listed in the table on page 18 are preferred. All of the disclosure in WO 02/070514 relating to IP receptor antagonists, is hereby incorporated herein by reference.

WO 02/070500 (F. Hoffman-La Roche AG) discloses alkoxycarbonylamino benzoic acid or alkoxycarbonylamino tetrazolyl phenyl derivatives which are IP receptor antagonists. Of these, the compounds listed in the table on page 24 are preferred. All of the disclosure in WO 02/070500 relating to IP receptor antagonists, is hereby incorporated herein by reference.

WO 02/40453 (F. Hoffman-La Roche AG) discloses substituted 2-phenylaminoimidazoline phenyl ketone derivatives which are IP receptor antagonists. Of these, the compounds listed in the table spanning pages 26-28 are preferred. All of the disclosure in WO 02/40453 relating to IP receptor antagonists, is hereby incorporated herein by reference.

WO 01/68591 (F. Hoffman-La Roche AG) discloses carboxylic acid derivatives which are IP receptor antagonists. Of these, the compounds listed in the table on page 40 are preferred. All of the disclosure in WO 01/68591 relating to IP receptor antagonists, is hereby incorporated herein by reference.

WO 01/10433 (Teijin, Inc.) discloses prostacyclin derivatives with an amino or amido group at the extremity of the α chain which may be IP receptor antagonists. All of the disclosure in WO 01/10433 relating to IP receptor antagonists, is hereby incorporated herein by reference.

WO 01/10445 (Teijin, Inc.) discloses 15(R)-isocarbacyclin and 15-deoxy-isocarbacyclin derivatives which may be IP receptor antagonists. All of the disclosure in WO 01/10445 relating to IP receptor antagonists, is hereby incorporated herein by reference.

WO 79/00744 (Research Corporation) discloses 6,9-thiaprostacyclin analogues and derivatives thereof which are stated as being antagonists for natural prostacyclin. All of the disclosure in WO 79/00744 relating to IP receptor antagonists, is hereby incorporated herein by reference.

Corsini et al (1987 and 1998) describe (5Z)-carbacyclin as a partial agonist of the PGI₂ receptor which also displays antagonistic properties (Corsini et al (1987) Br. J. Pharmac. 90, p225-261; Corsini et al (1988), Biomedica biochimica acta 1988, 47(10-11) pS104-7).

Corsini et al (1987) also mention that the PGI₂ analogue FCE 22176 ((5Z)-13,14-didehydro-20-methyl-carboprostacyclin) has been shown to be a competitive antagonist of PGI₂ on guinea pig trachea and atrium (Fassina et al, (1985) Eur. J. Pharmac. 113, p459-460).

Armstrong (1996, Pharmacol. Ther. 72(3), p171-191) in a review of platelet prostanoid receptors, mentions that the best lead in the search for an IP 25 antagonist has been the synthesis of 2-[3-[3-(4,5-diphenyl-2-oxazolyl)ethyl]phenoxy] acetic acid (BMY 42393) which is a partial agonist at the platelet IP receptor (Seiler et al (1994), Thromb Res. 74, p115-123).

For the avoidance of doubt, indomethacin is not an IP receptor antagonist according to the invention.

In an embodiment, the IP antagonist is an antibody.

The antibody may be monoclonal or polyclonal, but is preferably monoclonal. Given the cDNA and amino acid sequence of the human IP receptor (Genbank Accession Nos. NM_{—}000960 and NP_{—}000951, respectively) preparation of monoclonal antibodies, including humanised antibodies, is well within the ability of a person of average skill in the art.

Antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J. Hurrell (CRC Press, 1982), both of which are incorporated herein by reference.

Human IP receptor antibodies for use in the present invention can be raised against the intact IP protein or an antigenic polypeptide fragment thereof, which may be presented together with a carrier protein, such as an albumin, to an animal system (such as rabbit or mouse) or, if it is long enough (at least about 25 amino acids), without a carrier. Typically, the antigenic polypeptide fragment of the IP receptor is or comprises an extracellular portion of the IP receptor.

As used herein, the term “antibody” (Ab) or “monoclonal antibody” (Mab) is meant to include intact molecules as well as antibody fragments (such as, for example, Fab and F(ab′)2 fragments) which are capable of specifically binding to the IP receptor protein. Fab and F(ab′)2 fragments lack the Fc fragment of intact antibody, clear more rapidly from the circulation, and may have less non-specific tissue binding of an intact antibody (Wahl et al, J. Nucl. Med. 24:316-325 (1983)). Thus, these fragments are preferred. Alternatively, IP receptor binding fragments can be produced through the application of recombinant DNA technology or through synthetic chemistry. It is further appreciated that other antibody-like molecules may be used in the method of the inventions including, for example, antibody derivatives which retain their antigen-binding sites, synthetic antibody-like molecules such as single-chain Fv fragments (ScFv) and domain antibodies (dAbs), and other molecules with antibody-like antigen binding motifs.

The antibodies may be prepared by any of a variety of methods. For example, cells expressing the IP receptor or an antigenic fragment thereof can be administered to an animal in order to induce the production of sera containing polyclonal antibodies. In a preferred method, a preparation of IP receptor protein is prepared and purified to render it substantially free of natural contaminants. Such a preparation is then introduced into an animal in order to produce polyclonal antisera of greater specific activity.

In the most preferred method, the antibodies are monoclonal antibodies (or IP receptor binding fragments thereof). Such monoclonal antibodies can be prepared using hybridoma technology (Kohler et al, Nature 256:495 (1975); Kohler et al, Eur. J. Immunol 6:511 (1976); Kohler et al, Eur. J. Immunol. 6:292 (1976); Hammerling et al, in: Monoclonal Antibodies and T-Cell Hybridomas, Elsevier, N.Y., (1981) pp. 563-681). In general, such procedures involve immunising an animal (preferably a mouse) with a IP receptor antigen or, more preferably, with an IP receptor expressing cell. It is further preferred if the mouse is a transgenic mouse which is transgenic for the human Ig region, thus producing humanised antibodies.

It is preferred if the anti-IP antibodies are humanised antibodies, which are suitable for administration to humans without engendering an immune response by the human against the administered immunoglobulin (Ig). Humanised forms of antibodies may include chimaeric Igs, Ig chains or fragments thereof (such as Fv, Fab, Fab′, F(ab′)₂ or other antigen-binding subsequences of antibodies) that are principally comprised of the sequence of a human Ig, and contain minimal sequence derived from a non-human Ig.

Methods for producing chimaeric antibodies are known in the art. See, for a review, Morrison, Science 229:1202 (1985); Oi et al, BioTechniques 4:214 (1986); Cabilly et al, U.S. Pat. No. 4,816,567; Taniguchi et al, EP 171496;

Morrison et al, EP 173494; Neuberger et al, WO 8601533; Robinson et al, WO 870267 1; Boulianne et al, Nature 312:643 (1984); Neuberger et al, Nature 314:268 (1985).

Humanisation can be performed following the method of Winter and co-workers (Jones et al, Nature, 321:522-525 (1986); Riechmann et al, Nature, 332:323-327 (1988); Verhoeyen et al, Science, 239:1534-1536 (1988)), by importing rodent CDRs or CDR sequences into a human antibody. (See also U.S. Pat. No. 5,225,539). In some instances, Fv framework residues of the human Ig are replaced by corresponding non-human residues. Humanised antibodies can also comprise residues that are found neither in the recipient antibody nor in the imported CDR or framework sequences. In general, the humanised antibody will comprise substantially all of at least one, and typically two, variable domains, in which all or substantially all of the CDR regions correspond to those of a non-human Ig and all or substantially all of the framework regions are those of a human Ig consensus sequence. The humanised antibody optimally also will comprise at least a portion of an Ig constant region (Fc), typically that of a human Ig (Jones et al, 1986; Riechmann et al, 1988; and Presta, Curr. Op. Struct. Biol., 2:593-596 (1992)).

Humanised antibodies also include antibody molecules in which essentially the entire sequences of both the light chain and the heavy chain, including the CDRs, arise from human genes. Humanised monoclonal antibodies can be prepared by the trioma technique; the human B-cell hybridoma technique (see Kozbor, et al, 1983 Immunol Today 4: 72) and the EBV hybridoma technique to produce humanised monoclonal antibodies (see Cole, et al, 1985 In: Monoclonal Antibodies and Cancer Therapy, Alan R. Liss, Inc., pp. 77-96).

In addition, humanised antibodies can also be produced using techniques such as phage display libraries (Hoogenboom and Winter, J. Mol. Biol., 227:381 (1991); Marks et al, J. Mol. Biol., 222:581 (1991)).

Similarly, humanised antibodies can be made by introducing human Ig loci into transgenic animals, e.g., mice in which the endogenous Ig genes have been partially or completely inactivated. Upon challenge, antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al (Bio/Technology 10, 779-783 (1992)); Lonberg et al (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

The preferred embodiment of such a nonhuman animal is a mouse, and is termed the Xenomouse™ as disclosed in PCT publications WO 96/33735 and WO

This animal produces B cells that secrete fully human Igs. The antibodies can be obtained directly from the animal after immunisation with an immunogen of interest, as, for example, a preparation of a polyclonal antibody, or alternatively from immortalised B cells derived from the animal, such as hybridomas producing monoclonal antibodies. Additionally, the genes encoding the Igs with human variable regions can be recovered and expressed to obtain the antibodies directly, or can be further modified to obtain analogues of antibodies such as, for example, single chain Fv molecules.

A further method for producing a humanised antibody is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

Additional IP receptor antagonists may be identified using the screening methods described in EP 0 753 528 A1 (Ono Pharmaceutical Co.) and U.S. Pat. Nos. 5,728,808 and 6,365,360 (assigned to Merck Frosst Canada & Co.), all of which are incorporated herein by reference.

For the avoidance of doubt, by “antagonist of the IP receptor” we include an antagonist of PGI₂, which may be any PGI₂ antagonist that is suitable to be administered to the patient. The PGI₂ antagonists are preferably selective to PGI₂ and typically have a higher binding affinity for PGI₂ than for other molecules. Although antagonists with a higher affinity for PGI₂ than other molecules are preferred, antagonists with a lower affinity may also be used, but it may be necessary to use these at higher concentrations. Preferably, the PGI₂ antagonists bind reversibly to PGI₂.

Suitable PGI₂ antagonists may include extracellular fragments of the IP receptor.

As used herein, the term ‘antagonist’ covers all types of antagonism. (G-protein-coupled receptors (GPCRs) such as prostaglandin receptors are known to show inverse agonism which has the outcome of blocking a desired response. Thus a suitable IP antagonist for use in the present invention may be identified by measuring the binding of a radio-labelled IP agonist to PGI₂ with or without the purported antagonist. Secondly, IP antagonists may be identified in a functional assay eg by showing that the effect of an IP agonist on Ca²⁺ levels is modified in the presence of the antagonist. Thirdly IP antagonists may be identified by inhibition of epithelial cell growth in cell culture.

In an embodiment, the inhibitor of PGIS is an anti-PGIS antibody.

A suitable antibody may be the anti-PGIS antibody available as Catalogue No. CAY-160630 from Alexis Corporation (Nottingham, UK). This is a monoclonal antibody against bovine PGIS, which has 88% sequence homology with human PGIS.

Preferably, the anti-PGIS antibody is a humanised antibody as described above. Given the cDNA and protein sequence of human PGIS (Genbank Accession Nos. NM_{—}000961 and NP_{—}000952, respectively) preparation of monoclonal antibodies, including humanised antibodies, is well within the ability of a person of average skill in the art.

The compound U-51605 has been reported as being a PGIS inhibitor (Bayorh et al, ASGSB 2001 Annual Meeting Abstract No. 82).

Peryoxynitrite (PON) and the PON generator 3-morpholinosydnonimine N-ethylcarbamide (SIN-1) have been reported to inhibit PGIS by tyrosine nitration at the active site (Zou et al, 1998, Biochem. J. 336: 507-512).

Trans-2-phenylcyclopropylamine HCl has been reported as being a PGIS inhibitor (Gryglewski et al, Prostaglandins 1976, 12: 685; Hoyns & van Alphen, Doc. Ophthol. (Netherlands) 1981, 51: 225).

All of the patent and non-patent documents referred to herein that describe antagonists and inhibitors of the IP receptor or PGIS are incorporated herein, in their entirety, by reference.

EP Receptor Antagonists

PGE₂ mediates its effect on target cells through interaction with different isoforms of seven transmembrane G protein coupled receptors which belong to the rhodopsin family of serpentine receptors. Four main PGE₂ receptor subtypes have been identified (EP1, EP2, EP3 and EP4) which utilise alternate and in some cases opposing intracellular signalling pathways (Coleman et al, 1994). This diversity of receptors with opposing action may confer a homeostatic control on the action of PGE₂ that is released in high concentrations close to its site of synthesis (Ashby, 1998). To-date, the role of the different PGE₂ receptors, their divergent intracellular signalling pathways, as well as their respective target genes involved in mediating the effects of PGE₂ on normal or neoplastically transformed endometrial epithelial cells remain to be elucidated fully.

We have previously studied the expression of PGE synthase and of the EP2 and EP4 receptors across the menstrual cycle and have found that PGES expression is reduced during the late secretory phase and that EP4 expression is significantly higher in the late proliferative stage (Milne et al, (2001) J. Clin. Endocrinol. 86(9): 4453-4459). We have also previously found that PGES expression and PGE₂ synthesis are also up-regulated in pathological conditions of the uterus in humans. For example, in adenocarcinoma, expression of these factors was localised to the neoplastic epithelial cells of the uterine carcinoma tissues as well as the endothelial cells of the microvasculature. This is associated with an overexpression and signalling of the EP2 and EP4 receptors in the carcinoma tissue. We have previously suggested the use of an inhibitor of PGES or an EP2 or EP4 receptor antagonist in the treatment or prevention of a pathological condition of the uterus (PCT/GB02/004845 and PCT/GB02/004549).

In a further embodiment of the present invention, in addition to the at least one agent that is an antagonist of the IP receptor, the individual is also administered an inhibitor of PGES and/or an antagonist of EP2 or of EP4 (which term, for the avoidance of doubt, includes an antagonist or an EP2 receptor and an antagonist of an EP4 receptor, respectively).

In one embodiment of the invention, the individual is administered an inhibitor of PGES. It has been reported by Thoren & Jakobsson (2000) Eur. J Biochem. 267, 6428-6434 (incorporated herein by reference) that NS-398, sulindac sulphide and leukotriene C₄ inhibit PGES activity with IC₅₀ values of 20 μM, 80 μM and 5μM, respectively.

In a still further embodiment of the invention, the individual is administered an antagonist of an EP2 receptor or an antagonist of an EP4 receptor.

The prostaglandin EP2 receptor antagonist may be any suitable EP2 receptor antagonist. Similarly, the prostaglandin EP4 receptor antagonist may be any suitable EP4 receptor antagonist. By “suitable” we mean that the antagonist is one which may be administered to a patient. The receptor antagonists are molecules which bind to their respective receptors, compete with the natural ligand (PGE₂) and inhibit the initiation of the specific receptor-mediated signal transduction pathways. The receptor antagonists are typically selective to the particular receptor and typically have a higher binding affinity to the receptor than the natural ligand. Although antagonists with a higher affinity for the receptor than the natural ligand are preferred, antagonists with a lower affinity may also be used, but it may be necessary to use these at higher concentrations. Preferably, the antagonists bind reversibly to their cognate receptor. Typically, antagonists are selective for a particular receptor and do not affect the other receptor; thus, typically, an EP2 receptor antagonist binds the EP2 receptor but does not substantially bind the EP4 receptor, whereas an EP4 receptor antagonist binds the EP4 receptor but does not substantially bind the EP2 receptor. Preferably, the EP2 or EP4 receptor antagonist is selective for the particular receptor subtype. By this is meant that the antagonist has a binding affinity for the particular receptor subtype which is at least ten-fold higher than for at least one of the other EP receptor subtypes. Thus, selective EP4 receptor antagonists have at least a ten-fold higher affinity for the EP4 receptor than any of the EP1, EP2 or EP3 receptor subtypes.

It is particularly preferred that the EP2 or EP4 receptor antagonist is selective for its cognate receptor.

EP2 receptor antagonists include AH6809 (Pelletier et al (2001) Br. J. Phamacol. 132, 999-1008).

EP4 receptor antagonists include AH23848B (developed by Glaxo) and AH22921X (Pelletier et al (2001) Br. J. Pharmacol. 132, 999-1008. The chemical name for AH23848B is ([1alpha(z), 2beta5alpha]-(±)-7-[5-[[(1,1′-biphenyl)-4-yl]methoxy]-2-(4-morpholinyl)-3-oxo-cyclopentyl]-4-heptenoic s acid) (see Hillock & Crankshaw (1999) Eur. J. Pharmacol. 28, 99-108). EP4RA (Li (2000) Endocrinology 141, 2054-61) is an EP(4) -selective ligand (Machwate et al (2001) Mol. Pharmacol. 60: 36-41). The omega-substituted prostaglandin E derivatives described in WO 00/15608 (EP 1 114 816) (Ono Pharm Co Ltd) bind EP4 receptors selectively and may be EP4 receptor antagonists.

Peptides described in WO 01/42281 (Hopital Sainte-Justine) eg: IFTSYLECL (SEQ ID No 1), IFASYECL (SEQ ID No 2), IFTSAECL (SEQ ID No 3), IFTSYEAL (SEQ ID No 4), ILASYECL (SEQ ID No 5), IFTSTDCL (SEQ ID No 6), TSYEAL (SEQ ID No 7) (with 4-biphenyl alanine), TSYEAL (SEQ ID No 7) (with homophenyl alanine) are also described as EP4 receptor antagonists, as are some of the compounds described in WO 00/18744 (Fujisawa Pharm Co Ltd). The 5-thia-prostaglandin E derivatives described in WO 00/03980 (EP 1 097 922) (Ono Pharm Co Ltd) may be EP4 receptor antagonists.

EP4 receptor antagonists are also described in WO 01/10426 (Glaxo), WO 00/21532 (Merck) and GB 2 330 307 (Glaxo).

WO 00/21532 describes the following as EP4 receptor antagonists:

5-butyl-2,4-dihydro-4-[[2 ′-[N-(3-chloro-2-thiophenecarbonyl)sulfamoyl]biphenyl-4-yl]methyl]-2-{2-(trifluoromethyl)phenyl]-1,2,4-triazol-3-one potassium salt;

5-butyl-2,4-dihydro-4-[[2 ′-[N-(2-methyl-3-furoyl)sulfamoyl]biphenyl-4-yl]methyl]-2-{2-(trifluoromethyl)phenyl]-1,2,4-triazol-3-one;

5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one;

5-butyl-2,4-dihydro-4-[[2 ′-[N-(2-thiophenecarbonyl)sulfamoyl]biphenyl-4-yl]methyl]-2-{2-(trifluoromethyl)phenyl]-1,2,4-triazol-3-one;

5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.

GB 2 330 307 describes [1α(Z), 2β,5α]-(±)-7-[5-[[(1,1′-biphenyl)-4-yl]methoxy]-2-(4-morpholinyl)-3-oxocyclopentyl]-4-heptenoic acid and [1R[1α(z),2β,5α]]-(−)-7-[5-[[(1,1′-biphenyl)-4-yl]methoxy]-2-(4-morpholinyl)-3-oxocyclopentyl]-4-heptenoic acid.

WO 00/18405 (Pharmagene) describes the EP4 receptor antagonists AH22921 and AH23848 (which are also described in GB 2 028 805 and U.S. Pat. No. 4,342,756). WO 01/72302 (Pharmagene) describes further EP4 receptor antagonists, for example those described by reference to, and included in the general formula (I) shown on page 8 et seq.

In an embodiment, when an inhibitor of PGES and/or an antagonist of EP2 or of EP4 is administered to a patient in addition to the at least one agent that is an antagonist of the IP receptor, the dose of each compound may be the same as would be administered individually without reference to the other compound. Alternatively and preferably, lower doses may be administered.

All of the patents and other documents referred to herein that describe antagonists or inhibitors of EP2 or EP4 and PGES, are incorporated herein, in their entirety, by reference.

FP Receptor Antagonists

The FP prostaglandin receptor has been studied in a variety of tissues including the bovine corpus luteum (Sharif et al 1998, J. Pharmacol. Exp. Ther. 286: 1094-1102); human uterus (Senior et al 1992, Br. J. Pharmacol. 108: 501-506); rabbit jugular vein (Chen et al 1995, Br. J. Pharmacol. 116: 3035-3041); various human ocular tissues (Davis & Sharif 1999, J. Ocular Pharmacol. Ther. 15: 323-336); and in mouse Swiss 3T3 fibroblasts (Griffin et al 1997, J. Pharmacol. Exp. Ther. 281: 845-854); and in rat vascular smooth muscle cells (A7r5) (Griffin et al 1998, J. Pharmacol. Exp. Ther. 286: 411-418).

Potent, selective synthetic agonists at some prostaglandin receptors have been characterised in both in vitro and in vivo models (Coleman et al 1994, Pharmacol. Rev. 46: 205-229). For instance fluprostenol or its enantiomer (eg AL-5848) (Sharif et al 1999, J Pharm. Pharmacol., 51: 685-694) and cloprostenol (Coleman et al 1994; Sharif et al 1998) are potent and selective FP receptor agonists. Since most natural prostaglandins show rather limited selectivity for their preferred receptor among this receptor family, the few reported selective prostaglandin receptor agonists have been very valuable tools for discriminating discrete functional responses coupled to their respective receptors. However, conclusive identification of the particular receptors mediating prostaglandin-stimulated functional responses requires potent and selective antagonists (Kenakin 1996, Pharmacol. Rev. 48: 413:463).

The recent identification and commercial development of selective FP receptor agonists as potent and highly efficacious drugs for the treatment of elevated intraocular pressure (Bito 1997, Surv. Ophthalnol. 41 (Suppl. 22): S1-S14; Hellberg et al 1998, Invest. Ophthalmol. Vis. Sci. 39 (Suppl.): 1961) has considerably advanced our knowledge of FP receptor-coupled pharmacological actions. However, the function of the FP receptor is not fully understood, due in part to significant species differences in the tissue distribution of this receptor (Ocklind et al 1996, Invest. Ophthalmol. Vis. Sci. 37: 716-726; Davis & Sharif 1999; Sharif et al 1999).

Griffin et al (J. Pharmacol. Exp. Ther. 1999, 290: 1278-1284) reported the discovery of a selective FP receptor antagonist (AL-8810) of micromolar potency. Sharif et al (J. Pharm. Pharmacol. 2000, 52: 1529-1539) describe another analogue of PGF_{2α} (AL-3138; Ro-22-6641; 11-deoxy-16-fluoro PGF_{2α}) which is a partial agonist of low efficacy and which also functions as an FP receptor antagonist. AL-3138 being a relatively selective agent may be a valuable FP receptor antagonist tool for investigating the specific function of the FP receptor in various biological systems.

We have also previously shown that expression of the PGF_{2α} receptor in the uterus across the menstrual cycle demonstrates higher level of the receptor during the proliferative phase of the endometrium compared with other stages. Expression in uterine carcinoma tissue is significantly elevated compared with normal uterine tissue. Using an endometrial epithelial cell line, we have demonstrated that PGF_{2α} induces proliferation of epithelial cells. This proliferation can be inhibited by using specific inhibitors of the PLC signalling pathway. These observations demonstrate the possibility of antagonising the PGF_{2α} (FP) receptor to combat pathological conditions of the uterus, such as to reduce the proliferation of epithelial cells in uterine carcinoma, as we have previously suggested (GB 0208785.6). Antagonists of the FP receptor have also been suggested for treating or preventing premature delivery of a foetus and dysmenorrhoea, acting by the mechanism of relaxation of smooth muscle (WO 99/32640 and WO 00/17348). We have also suggested using antagonists of the FP receptor to treat menorrhagia (GB 0208783.1).

In a further embodiment of the present invention, in addition to the at least one agent that is an antagonist of the IP receptor, and optionally an inhibitor of

PGES and/or an antagonist of EP2 or of EP4, the individual is also administered at least one agent that is an antagonist of the FP receptor.

Typically, the agent is one which prevents or disrupts PGF_{2α}-mediated signalling of the FP receptor. Preferably, an agent that is an antagonist of the FP receptor prevents or reduces the binding of PGF_{2α} to the FP receptor. Alternatively or additionally, the agent may affect the interaction between PGF_{2α} and the FP receptor, or the interaction between the FP receptor and the associated G_{αq} protein, thus inhibiting or disrupting the PGF_{2α}-FP mediated signal transduction pathway.

In one preferred embodiment, the agent that is an antagonist of the FP receptor may be an antagonist of the FP receptor. FP receptor antagonists are typically molecules which bind to the FP receptor, compete with the binding of the natural ligand PGF_{2α}, and inhibit or disrupt the PGF_{2α}-FP mediated signal transduction pathway.

In one preferred embodiment, antagonising the FP receptor includes occupying the PGF_{2α} binding site on the prostaglandin receptor, such that the natural ligand (PGF_{2α}) is prevented from binding in a mode that would result in its normal mode of signalling via Gq/Gq_{II} through inosityl phosphate and subsequent mobilisation of intracellular calcium.

Alternatively, the receptor antagonist may be a molecule which binds to the FP receptor without preventing PGF_{2α} binding thereto, but which disrupts the interaction between PGF_{2α} and the FP receptor, thus inhibiting or disrupting PGF_{2α}-FP mediated signal transduction pathway.

Further alternatively, the FP receptor antagonist may be a molecule which binds to the FP receptor and which disrupts the interaction between the FP receptor and the associated G_{αaq} protein, thus inhibiting or disrupting FP mediated signal transduction pathway.

For the avoidance of doubt, by “antagonist of the FP receptor” we include an antagonist of PGF_{2α}. PGF_{2α} antagonists are typically molecules which bind to PGF_{2α} and prevent or reduce PGF_{2α} binding to its receptor, which inhibits or disrupts the PGF_{2α}-FP mediated signal transduction pathway. This is often termed the ‘soluble receptor’ approach in which typically either a part of the receptor or an antibody binds to PGF_{2α}.

Alternatively, the PGF_{2α} antagonist may be a molecule which binds to PGF_{2α} without preventing or reducing the binding of PGF_{2α} to the FP receptor, but which disrupts the interaction between PGF_{2α} and the FP receptor such that the PGF_{2α}-FP mediated signal transduction pathway is inhibited or disrupted. This could be a molecule which binds in a covalent fashion to PGF_{2α} and has no effect on binding potency but effects the G-protein/IP/Ca²⁺ mechanisms.

The receptor antagonists are typically selective to the particular receptor and preferably have an equal or higher binding affinity to the FP receptor than does PGF_{2α}. Although antagonists with a higher affinity for the receptor than the natural ligand are preferred, antagonists with a lower affinity may also be used, but it may be necessary to use these at higher concentrations. Preferably, the antagonists bind reversibly to the FP receptor. Preferably, antagonists are selective for a particular receptor and do not affect other receptors; thus, typically, an FP receptor antagonist binds the FP receptor but does not substantially bind any other receptor.

The peptides listed in Table 1 are reported to be antagonists of the FP receptor that disrupt the interaction between the FP receptor and the associated G_{αq} protein (WO 99/32640 and WO 00/17438). The amino acids are indicated according to the standard IUPAC single letter convention, and X is cyclohexyl alanine. Lower case letters indicate L-amino acids and capital letters indicate D-amino acids. (For the avoidance of doubt, it is only for the peptides in Table 1 where this nomenclature is used. All other peptides, unless indicated to the contrary, are comprised of L-amino acids.) All of the disclosure in WO 99/32640 and WO 00/17438 relating to specific peptides as FP receptor antagonists, is hereby incorporated herein by reference.

TABLE 1

| | | |
|---|---|---|
| PCP-1 | rvkfksqqhrqgrshhlem | (SEQ ID No 8) |
| PCP-2 | rkavlknlyklasqccgvhvislhiw | (SEQ ID No 9) |
| | elssiknslkvaaisespvaeksast | |
| PCP-3 | clseeakearrindeierqlrrdkrd | (SEQ ID No 10) |
| | arre-NH₂ | |
| PCP-4 | kdtilqlnlkeynlv-NH₂ | (SEQ ID No 11) |
| PCP-8 | ilghrdyk | (SEQ ID No 12) |
| PCP-10 | wedrfyll | (SEQ ID No 13) |
| PCP-13 | ILGHRDYK | (SEQ ID No 14) |
| PCP-14 | YQDRFYLL | (SEQ ID No 15) |
| PCP-13.7 | ILAHRDYK | (SEQ ID No 16) |
| PCP-13.8 | ILaHRDYK | (SEQ ID No 17) |
| PCP-13.11 | ILGFRDYK | (SEQ ID No 18) |
| PCP-13.13 | ILGHKDYK | (SEQ ID No 19) |
| PCP-13.14 | ILGHRNYK | (SEQ ID No 20) |
| PCP-13.18 | ILGHQDYK | (SEQ ID No 21) |
| PCP-13.20 | ILGHRDY-amide | (SEQ ID No 22) |
| PCP-13.21 | ILGHRDYK-amide | (SEQ ID No 23) |
| PCP-13.22 | ILGWRDYK | (SEQ ID No 24) |
| PCP-13.24 | ILGXRDYK | (SEQ ID No 25) |
| PCP-15 | SNVLCSIF | (SEQ ID No 26) |

When the antagonist comprises a peptide, such as those mentioned in Table 1, the antagonist may also comprise protein fusions or peptidomimetics thereof.

PGF_{2α} dimethyl amide, obtained from Cayman Chemical, Ann Arbor, Mich., USA was reported to be a PGF_{2α} receptor antagonist (Arnould et al, (2001) Am. J. Pathol., 159(1): 345-357).

AL-8810 ((5Z, 13E)-(9S, 11S,15R)-9,15-dihydroxy-11-fluoro-15-(2-indanyl)-16, 17, 18, 19, 20-pentanor-5,13-prostadienoic acid) obtained from Alcon Research was reported to be a weak partial agonist of the PGF_{2α} receptor and a highly selective antagonist of the PGF_{2α} receptor. AL-8810 was reported not to significantly inhibit functional responses of prostaglandin receptors TP, DP, EP2 or EP4 at high 10 μM concentration (Griffin et al, (1999) J. Pharmacol. Exp. Ther., 260(3): 1278-1284).

AL-3138 (1 1-deoxy-16-fluoro PGF_{2α}) was reported to be a weak partial agonist of the PGF_{2α} receptor, and also a highly selective antagonist of the PGF_{2α} receptor (Sharifet al, (2000) J. Pharm. Pharmacol., 52(12): 1529-1539).

Phloretin was reported to be a PGF_{2α} receptor antagonist (Kitanaka et al (1993) J. Neurochem. 60(2): 704-708).

The sulfonylurea glibenclamide was reported to be a PGF_{2α} receptor antagonist (Delaey and Van de Voorde (1995), Eur. J. Pharmacol. 280(2): 179-184). The sulfonylureas tolbutamide and tolazamide were reported to be very weak antagonists of the FP receptor. (Sharif et al (2000) J. Pharm. Pharmacol., 52(12): 1529-1539).

PGF_{2α} dimethyl amine was reported to be a PGF_{2α} receptor antagonist (Stinger et al (1992), J. Pharmacol. Exp. Ther., 220: 521-525).

(E)-5-[[[(3-pyridinyl)[3-(trifluoromethyl) phenyl]methylen]amino]oxy]pentanoic acid, also known as ridogrel, obtained from Jannsen Pharmaceutica, was reported to be a PGF_{2α} receptor antagonist (Jannsens et al, (1990), Thrombosis and Haemostasis, 64(1): 91-96).

The compound PHG113 was reported to be a selective PGF_{2α} receptor antagonist (Quiniou et al, (2001) Pediatric Research, 49(2): 452A.

EP-128479 describes pyrazolyl-methyl-ergoline derivatives which are reported to be PGF_{2α} receptor antagonists. All the disclosure in EP-128479 relating to pyrazolyl-methyl-ergoline derivatives as PGF_{2α} receptor inhibitors, is hereby incorporated herein by reference.

The PGF_{2α} antagonists (which as noted above are included in the term FP receptor antagonist) are preferably selective to PGF_{2α} and typically have a higher binding affinity for PGF_{2α} than for other molecules. Although antagonists with a higher affinity for PGF_{2α} than other molecules are preferred, antagonists with a lower affinity may also be used, but it may be necessary to use these at higher concentrations. Preferably, the PGF_{2α} antagonists bind reversibly to PGF_{2α}.

PGF_{2α} antagonists include anti-PGF_{2α} antibodies such as rabbit polyclonal anti-PGF_{2α} antibodies from Oxford Biomedical Research, Inc., Oxford, UK (Arnould et al, Am. J. Pathol. 2001 159(1): 345-357). Arnould et al state that, according to the manufacturer, the specificity of the antibody is very high and the cross-reactivity with other prostanoid derivatives is <1%.

JP 04077480; JP 08176134; JP 01199958; JP 01050818; and JP 63083081 each describe phthalide derivatives that are reported to be PGF_{2α} inhibitors. All the disclosure in JP 04077480; JP 08176134; JP 01199958; JP 01050818; and JP 63083081 relating to phthalide derivatives as PGF_{2α} inhibitors, is hereby incorporated herein by reference.

WO 91/13875 describes (iso) quinoline sulphonamide compounds which are reported to be PGF_{2α} inhibitors. All the disclosure in WO 91/13875 relating to (iso) quinoline sulphonamide compounds as PGF_{2α} inhibitors, is hereby incorporated herein by reference.

Some of the compounds reported as being inhibitors or antagonists of PGF_{2α} may, in fact, be antagonists of the PGF_{2α} (FP) receptor, as used and defined herein. References to such compounds as inhibitors or antagonists of PGF_{2α} should therefore be considered as references to FP receptor antagonists.

All of the patents and other documents referred to herein that describe FP antagonists or inhibitors, are incorporated herein, in their entirety, by reference.

COX-2 Inhibitors

We have previously shown that cyclooxygenase-2 (COX-2) synthesis is up-regulated in pathological conditions of the uterus in humans. For example, in adenocarcinoma of the human uterus, expression was localised to the neoplastic epithelial cells of the uterine carcinoma tissues as well as the endothelial cells of the microvasculature. We have previously suggested the use of an inhibitor of a COX-2 inhibitor, typically in conjunction with a PGES or an EP2 or EP4 receptor antagonist, in the treatment or prevention of a pathological condition of the uterus (PCT/GB02/004549).

In a further embodiment of the present invention, in addition to the at least one agent that is an antagonist of the IP receptor, and optionally an inhibitor of PGES and/or an antagonist of EP2 or of EP4, and also optionally an agent that is an antagonist of the FP receptor, the individual is also administered a COX-2 inhibitor.

Preferably the inhibitor is selective for COX-2.

The compound may selectively inhibit COX-2 function at any level. Suitably, the compound selectively inhibits COX-2 enzyme activity.

By “selectively inhibits COX-2 enzyme activity” we mean that the compound preferably inhibits COX-2 in preference to other cyclooxygenase enzymes, in particular in preference to COX-1. The COX-1 gene and the sequence of its polypeptide product are described in Yokoyama and Tanabe (1989) Biochem. Biophys. Res. Comm. 165, 888-894 incorporated herein by reference. COX-1 is also called PGHS-1. The COX-2 gene and the sequence of its polypeptide product are described in O'Banion et al (1991) J. Biol. Chem. 266, 23261-23267 incorporated herein by reference. COX-2 is also called PGHS-2.

Conveniently, the compound which selectively inhibits COX-2 enzyme activity is at least ten times better at inhibiting COX-2 than COX-1; preferably it is at least fifty times better; preferably it is at least one hundred times better; still more preferably it is at least one thousand times better and in greater preference it is at least ten thousand times better.

It is most preferred if the COX-2 inhibitor compound has substantially no inhibitory activity against the COX-1 enzyme.

Conveniently, the compound selectively inhibits COX-2 enzyme production. The compound may, for example, selectively prevent transcription of the COX-2 or it may selectively prevent translation of the COX-2 message.

By “selectively inhibits COX-2 enzyme production” we mean that the compound preferably inhibits the production of COX-2 in preference to other cyclo-oxygenases, in particular in preference to the production of COX-1.

Conveniently, the compound which selectively inhibits COX-2 enzyme production is at least ten times better at inhibiting COX-2 production than COX-1 production; preferably it is at least fifty times better; more preferably it is at least one hundred times better; more preferably still it is at least one thousand times better; and in greater preference it is at least ten thousand times better.

It is most preferred if the compound has substantially no inhibitory activity against COX-1 enzyme production.

A particularly preferred embodiment is wherein the COX-2 inhibitor compound is any one of nimesulide, 4-hydroxynimesulide, flosulide, and meloxicam.

Nimesulide is N-(4-nitro-2-phenoxyphenyl) methanesulfonamide (also called 4-nitro-2-phenoxymethanesulfonanilide). Nimesulide is 100-fold more specific for COX-2 inhibition than for COX-1 inhibition. Nimesulide is manufactured by Boehringer.

Flosulide is 6-(2,4-difluorophenoxy)-5-methyl sulphonylamino-1-indanone (also known as N-6-(2,4-difluorophenoxy)-1-oxo-indan-5-yl methane-sulphonamide). Flosulide is 1000-fold more specific for COX-2 inhibition than for COX-1 inhibition. Flosulide is manufactured by Ciba Geigy.

Meloxicam is 4-hydroxy-2-methyl-N-(5-methyl-2-thiazolyl)-2H-1,2-benzothiazine-3-carboxamide 1,1-dioxide. Meloxicam is 1000-fold more specific for COX-2 inhibition than for COX-1 inhibition. Meloxicam is manufactured by Boehringer.

The synthesis of nimesulide is well known and is described in U.S. Pat. No. 3,840,597; the synthesis of flosulide is well known and is described in GB 2 092 144; and the synthesis of meloxicam is well known and is described in U.S. Pat. No. 4,233,299.

Other COX-2-specific inhibitors which may be useful in the practice of the invention include:

L 475 L337 which is 500-fold more specific for COX-2 inhibition than for COX-1 inhibition. This is manufactured by Merck Frost.

Vioxx, sold by Merck, is also a suitable COX-2 inhibitor.

SC 58125 Celecoxib which is 100-fold more specific for COX-2 inhibition than for COX-1 inhibition. Celecoxib is manufactured by Searle.

NS 398 which is manufactured by Taisho and which is very highly selective for COX-2.

DuP 697, which is COX-2-selective and is manufactured by DuPont.

Nimesulide, flosulide and meloxicam are COX-2 enzyme inhibitors, probably competitive inhibitors.

All of the patents and other documents referred to herein that describe COX-2 inhibitors, are incorporated herein, in their entirety, by reference.

It is appreciated that one or two or more agents that are antagonists of the IP receptor and/or PGIS inhibitors may be administered to the patient.

Typically, the agent(s) that is an antagonist of the IP receptor and/or PGIS inhibitor(s) is (are) administered in a quantity and frequency such that an effective dose is delivered to at least 90% of the IP receptors (ED₉₀). The potency of the molecule would dictate the dose, as would the formulation and route of administration.

Optionally, other treatment agents may also be administered to the patient, as described above. These may also be considered treatment agents of the invention. It will also be appreciated that when more than one treatment agent is administered to the patient, they may be administered sequentially or in combination.

The treatment agent(s) are administered in an effective amount to combat the undesired pathological condition of the uterus. Thus, the treatment agents may be used to alleviate symptoms (ie are used palliatively), or may be used to treat the condition, or may be used prophylactically to prevent the condition. The treatment agent may be administered by any suitable route, and in any suitable form.

The aforementioned treatment agents for use in the invention or a formulation thereof may be administered by any conventional method including oral and parenteral (eg subcutaneous or intramuscular) injection. The treatment may consist of a single dose or a plurality of doses over a period of time. The dose to be administered is determined upon consideration of age, body weight, mode of administration, duration of the treatment and pharmacokinetic and toxicological properties of the treatment agent or agents. The treatment agents are administered at a dose (or in multiple doses) which produces a beneficial therapeutic effect in the patient. Typically, the treatment agents are administered at a dose the same as or similar to that used when the treatment agent is used for another medical indication. In any event, the dose suitable for treatment of a patient may be determined by the physician.

Whilst it is possible for a treatment agent of the invention to be administered alone or in combination with other treatment agents, it is preferable to present it or them as a pharmaceutical formulation, together with one or more acceptable carriers. The carrier(s) must be “acceptable” in the sense of being compatible with the treatment agent of the invention and not deleterious to the recipients thereof Typically, the carriers will be water or saline which will be sterile and pyrogen free.

The formulations may conveniently be presented in unit dosage form and may be prepared by any of the methods well known in the art of pharmacy. Such methods include the step of bringing into association the treatment agent or agents with the carrier which constitutes one or more accessory ingredients. In general the formulations are prepared by uniformly and intimately bringing into association the active ingredient (ie treatment agent or agents) with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier. Buccal administration is also preferred.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

Certain of the treatment agents are proteins or peptides. Proteins and peptides may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The protein and peptide can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy systems can also be employed for the administration of proteins and peptides. A device which delivers a pulsed electric field to cells increases the permeability of the cell membranes to the drug, resulting in a significant enhancement of intracellular drug delivery.

Proteins and peptides can be delivered by electroincorporation which occurs when small particles of up to 30 microns in diameter on the surface of the skin experience electrical pulses identical or similar to those used in electroporation. In electroincorporation, these particles are driven through the stratum corneum and into deeper layers of the skin. The particles can be loaded or coated with drugs or genes or can simply act as “bullets” that generate pores in the skin through which the drugs can enter.

An alternative method of protein and peptide delivery is the ReGel injectable system that is thermo-sensitive. Below body temperature, ReGel is an injectable liquid while at body temperature it immediately forms a gel reservoir that slowly erodes and dissolves into known, safe, biodegradable polymers. The treatment agent is delivered over time as the biopolymers dissolve.

Protein and peptide pharmaceuticals can also be delivered orally. The process employs a natural process for oral uptake of vitamin B₁₂ in the body to co-deliver proteins and peptides. By riding the vitamin B₁₂ uptake system, the protein or peptide can move through the intestinal wall. Complexes are synthesised between vitamin B₁₂ analogues and the drug that retain both significant affinity for intrinsic factor (IF) in the vitamin B₁₂ portion of the complex and significant bioactivity of the drug portion of the complex.

Proteins and polypeptides can be introduced to cells by “Trojan peptides”.

These are a class of polypeptides called penetratins which have translocating properties and are capable of carrying hydrophilic compounds across the plasma membrane. This system allows direct targeting of oligopeptides to the cytoplasm and nucleus, and may be non-cell type specific and highly efficient. See Derossi et al (1998), Trends Cell Biol 8, 84-87.

The treatment agents or formulations may also be administered transdermally, eg as a patch, gel, lotion, cream or oil.

It is preferred if the treatment agent (or agents) is administered orally.

It is further preferred if the treatment agent (or agents) or formulation thereof is administered to the female reproductive system. For example, the treatment agent(s) may suitably be administered intravaginally using, for example, a gel or cream or paste or foam or spray or pessary or vaginal ring or tampon. The treatment agent may also advantageously be administered by intrauterine delivery, for example using methods well known in the art such as an intrauterine device.

Typically, the gel or cream is one which is formulated for administration to the vagina. It may be oil based or water based. Typically, the treatment agent(s) is present in the cream or gel in a sufficient concentration so that an effective amount is administered in a single (or in repeated) application.

Typically, the vaginal ring comprises a polymer which formed into a “doughnut” shape which fits within the vagina. The treatment agent (or agents) is present within the polymer, typically as a core, which may dissipate through the polymer and into the vagina and/or cervix in a controlled fashion. Vaginal rings are known in the art.

Typically, the tampon is impregnated with the treatment agent (or agents) and that a sufficient amount of the treatment agent (or agents) is present in the tampon.

Typically, the intrauterine device is for placing in the uterus over extended periods of time, such as between one and five years. Typically, the intrauterine device comprises a plastic frame, often in the shape of a “T” and contains sufficient of the treatment agent(s) to be released over the period of use. The agent is generally present within or encompassed by a slow-release polymer which forms part of the device, such as in the form of a “sausage” of agent which wraps around the long arm of the “T” which is typically covered with a controlled-release membrane. Intrauterine devices are known in the art.

A second aspect of the invention provides use of at least one agent that is an antagonist of the IP receptor and/or a PGIS inhibitor, in the manufacture of a medicament for combating a pathological condition of the uterus in a female individual.

In an embodiment of this aspect of the invention, the female individual is administered at least one agent that is an antagonist of the IP receptor and/or a PGIS inhibitor. Typically she is administered the at least one agent that is an antagonist of the IP receptor and/or a PGIS inhibitor at the same time as the medicament. Alternatively, the female may have been (or will be) administered the at least one agent that is an antagonist of the IP receptor and/or a PGIS inhibitor before (or after) receiving the medicament.

It is appreciated that in this and all subsequent aspects of the invention, preferences for an agent that is an antagonist of the IP receptor, and preferences for a PGIS inhibitor, are as described previously with respect to the first aspect of the invention.

It is also appreciated that in this and all subsequent aspects of the invention, preferences for the pathological condition of the uterus to be combated are as described previously with respect to the first aspect of the invention.

In an embodiment, the female individual is administered any one or two or more of an inhibitor of PGES and/or an antagonist of EP2 or EP4, an agent that is an antagonist of the FP receptor, and a COX-2 inhibitor. Typically the female is administered the any one or two or more of these additional agents at the same time as the medicament. Alternatively, the female may have been (or will be) administered the any one or two or more of these additional agents before (or after) receiving the medicament containing the at least one agent that is an antagonist of the IP receptor and/or a PGIS inhibitor.

Further alternatively, if any two or more of an inhibitor of PGES and/or an antagonist of EP2 or EP4, an agent that is an antagonist of the FP receptor, and a

COX-2 inhibitor are administered to the female, they may be administered separately. Thus, for example, a COX-2 inhibitor may be administered before receiving the medicament, and an inhibitor of PGES and/or an antagonist of EP2 or EP4 may be administered together with the medicament.

It is appreciated that in this and all subsequent aspects of the invention, preferences for an inhibitor of PGES and/or an antagonist of EP2 or EP4 are as described previously with respect to the first aspect of the invention.

It is also appreciated that in this and all subsequent aspects of the invention, preferences for an agent that is an antagonist of the FP receptor are as described previously with respect to the first aspect of the invention.

It is further appreciated that in this and all subsequent aspects of the invention, preferences for a COX-2 inhibitor are as described previously with respect to the first aspect of the invention.

A third aspect of the invention provides use of a combination of at least one agent that is an antagonist of the IP receptor and/or a PGIS inhibitor, and an inhibitor of PGES and/or an antagonist of EP2 or EP4, in the manufacture of a medicament for treating or preventing a pathological condition of the uterus in a female individual.

In an embodiment, the female individual is administered one or more of an agent that is an antagonist of the FP receptor, and a COX-2 inhibitor. In this case, typically the female is administered one or more of these additional agents at the same time as the medicament, although the female may have been (or will be) administered one or more of these additional agents before (or after) receiving the medicament.

A fourth aspect of the invention provides use of a combination of at least one agent that is an antagonist of the IP receptor and/or a PGIS inhibitor, and an agent is an antagonist of the FP receptor, in the manufacture of a medicament for treating or preventing a pathological condition of the uterus in a female individual.

In an embodiment, the female individual is administered one or more of an inhibitor of PGES and/or an antagonist of EP2 or EP4 and a COX-2 inhibitor. In this case, typically the female is administered one or more of these additional agents at the same time as the medicament, although the female may have been (or will be) administered one or more of these additional agents before (or after) receiving the medicament.

A fifth aspect of the invention provides use of a combination of at least one agent that is an antagonist of the IP receptor and/or a PGIS inhibitor, and a COX-2 inhibitor, in the manufacture of a medicament for treating or preventing a pathological condition of the uterus in a female individual.

In an embodiment, the female individual is administered one or more of an inhibitor of PGES and/or an antagonist of EP2 or EP4, and an agent that is an antagonist of the FP receptor. In this case, typically the female is administered one or more of these additional agents at the same time as the medicament, although the female may have been (or will be) administered one or more of these additional agents before (or after) receiving the medicament.

A sixth aspect of the invention provides use of a combination of at least one agent that is an antagonist of the IP receptor and/or a PGIS inhibitor, and an inhibitor of PGES and/or an antagonist of EP2 or EP4, and an agent that is an antagonist of the FP receptor, in the manufacture of a medicament for treating or preventing a pathological condition of the uterus in a female individual.

In an embodiment, the female individual is administered a COX-2 inhibitor. In this case, typically the female is administered the COX-2 inhibitor at the same time as the medicament, although the female may have been (or will be) administered the COX-2 inhibitor before (or after) receiving the medicament.

A seventh aspect of the invention provides use of at least one agent that is an antagonist of the IP receptor and/or a PGIS inhibitor, and an inhibitor of PGES and/or an antagonist of EP2 or EP4, and a COX-2 inhibitor, in the manufacture of a medicament for treating or preventing a pathological condition of the uterus in a female individual.

In an embodiment, the female individual is administered an agent that is an antagonist of the FP receptor. In this case, typically the female is administered the agent that is an antagonist of the FP receptor at the same time as the medicament, although the female may have been (or will be) administered the agent that is an antagonist of the FP receptor before (or after) receiving the medicament.

An eighth aspect of the invention provides use of at least one agent that is an antagonist of the IP receptor and/or a PGIS inhibitor, and an agent that is an antagonist of the FP receptor, and a COX-2 inhibitor, in the manufacture of a medicament for treating or preventing a pathological condition of the uterus in a female individual.

In an embodiment, the female individual is administered an inhibitor of PGES and/or an antagonist of EP2 or EP4. In this case, typically the female is administered the inhibitor of PGES and/or an antagonist of EP2 or EP4 at the same time as the medicament, although the female may have been (or will be) administered the inhibitor of PGES and/or an antagonist of EP2 or EP4 before (or after) receiving the medicament.

A ninth aspect of the invention provides use of a combination of at least one agent that is an antagonist of the IP receptor and/or a PGIS inhibitor, an inhibitor of PGES and/or an antagonist of EP2 or EP4, an agent that is an antagonist of the FP receptor, and a COX-2 inhibitor, in the manufacture of a medicament for treating or preventing a pathological condition of the uterus in a female individual.

A tenth aspect of the invention provides use of any one or two or more of an inhibitor of PGES and/or an antagonist of EP2 or EP4, an agent that is an antagonist of the FP receptor, and a COX-2 inhibitor, in the manufacture of a medicament for treating or preventing a pathological condition of the uterus in a female individual, wherein the individual is administered at least one agent that is an antagonist of the IP receptor and/or a PGIS inhibitor. In this case, typically the female is administered the at least one agent that is an antagonist of the IP receptor and/or the PGIS inhibitor at the same time as the medicament, although the female may have been (or will be) administered the at least one agent that is an antagonist of the IP receptor and/or the PGIS inhibitor before (or after) receiving the medicament.

In a preferred embodiment of any of the second to tenth aspects of the invention, the medicament is formulated to be administered via a vaginal ring or a tampon or an intrauterine device.

An eleventh aspect of the invention provides a composition comprising at least one agent that is an antagonist of the IP receptor and/or a PGIS inhibitor, and any one or two or more of an inhibitor of PGES and/or an antagonist of EP2 or EP4, an agent that is an antagonist of the FP receptor, and a COX-2 inhibitor.

A twelfth aspect of the invention provides a composition comprising at least one agent that is an antagonist of the IP receptor and/or a PGIS inhibitor, and any one or two or more of an inhibitor of PGES and/or an antagonist of EP2 or EP4, an agent that is an antagonist of the FP receptor, and a COX-2 inhibitor, for use in medicine.

A thirteenth aspect of the invention provides a pharmaceutical composition comprising at least one agent that is an antagonist of the IP receptor and/or a PGIS inhibitor, and any one or two or more of an inhibitor of PGES and/or an antagonist of EP2 or EP4, an agent that is an antagonist of the FP receptor, and a COX-2 inhibitor, and a pharmaceutically acceptable carrier.

A fourteenth aspect of the invention provides a vaginal ring or a tampon or an intrauterine device comprising at least one agent that is an antagonist of the IP receptor and/or a PGIS inhibitor, and any one or two or more of an inhibitor of PGES and/or an antagonist of EP2 or EP4, an agent that is an antagonist of the PP receptor, and a COX-2 inhibitor.

The invention will now be described in more detail by reference to the following Figures and Examples.

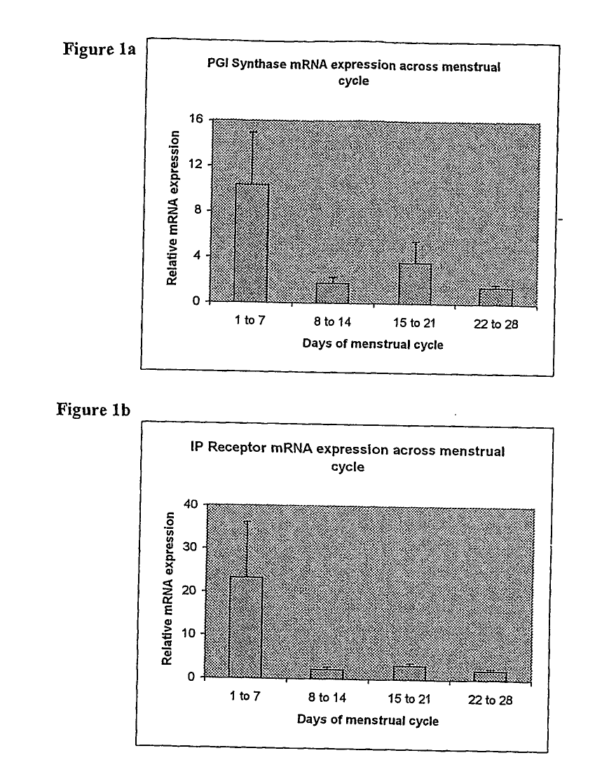

FIG. 1a: Variation in PGI synthase mRNA expression in endometrial biopsies from across the menstrual cycle. The relative expression was significantly lower in the late proliferative phase of the cycle (1.74±0.51; n=7; p<0.05) compared with the early proliferative phase (10.36±4.58; n=6). Expression was lower in the early secretory (3.56±1.89; n=17; p<0.06) and late secretory (1.42±0.29; n=4; p=0.068) phases.

FIG. 1b: Temporal variation in IP receptor mRNA expression in endometrial biopsies from across the menstrual cycle. The relative expression was significantly lower in the late proliferative phase (2.09±0.57; n=7; p<0.01), early secretory phase (3.16±0.56; n=17; p<0.005) and late secretory phase (2.08±0.55; n=5; p<0.01) compared with the early proliferative phase (23.28±12.8; n=6).

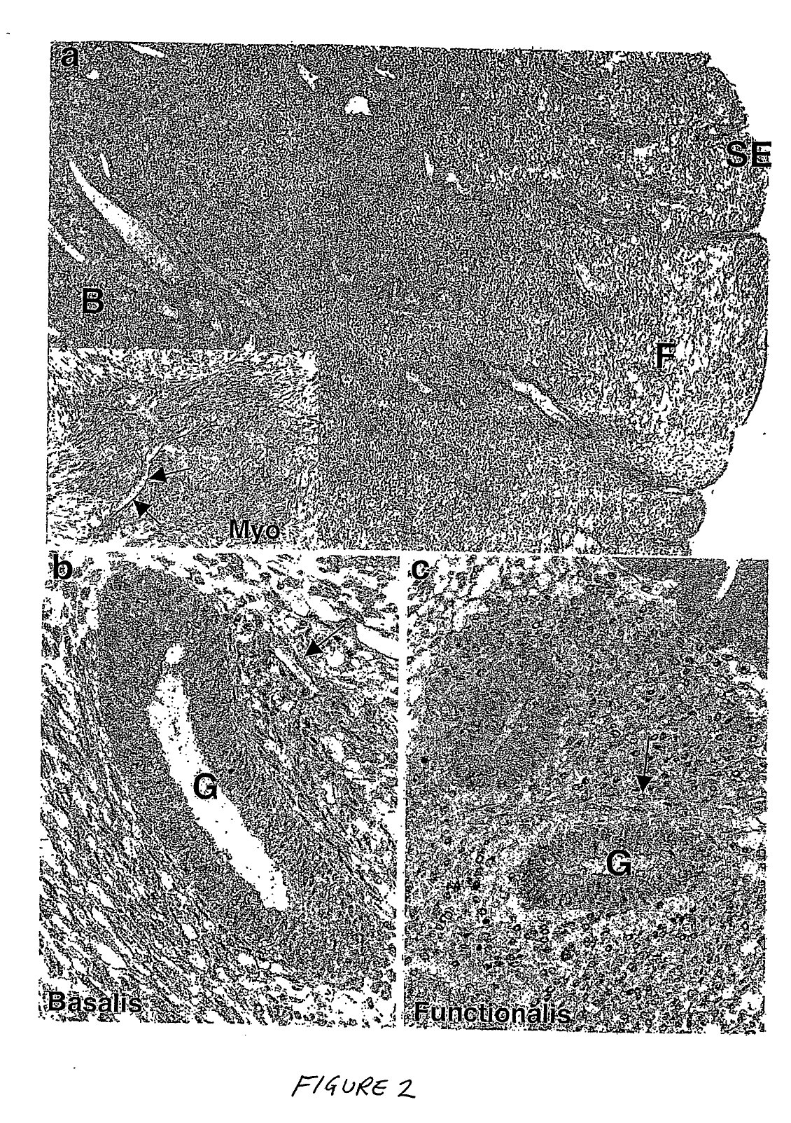

FIG. 2: Immunohistochemical localisation of PGI synthase in full-thickness endometrial tissue collected in the early proliferative, late proliferative (b) and early secretory (a) phases of the menstrual cycle. Strong staining was seen in the glandular epithelial cells (G) in basalis (B) and functionalis (F) layers and in the surface epithelium (SE). Stromal staining was seen in the functionalis layer (c) and the basalis layer (b). Strong staining was present in smooth muscle cells in the myometrium (Myo) and endothelial reactivity was present in blood vessels in all layers (arrowed in inset to a, b and c).

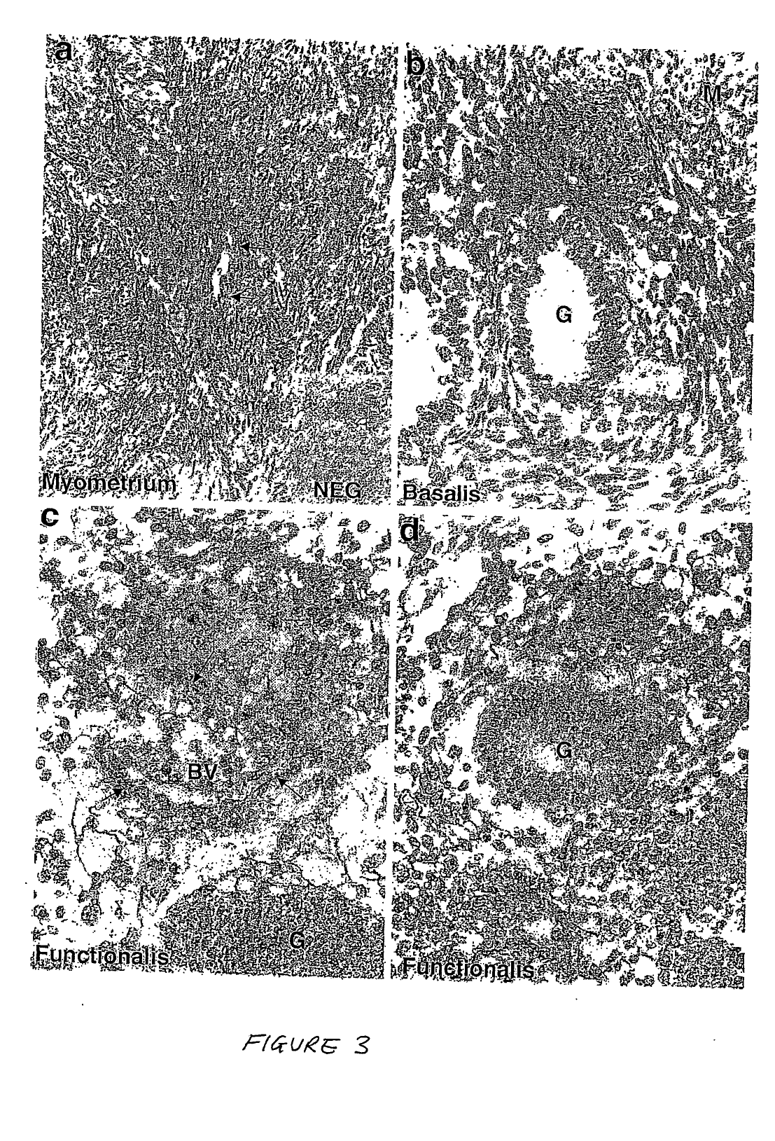

FIG. 3: In situ hybridisation of IP receptor probe to sections of human endometrial tissue collected in the mid proliferative phase of the menstrual cycle. IP receptor was expressed in the myometrium (a) with reactivity present in smooth muscle cells and blood vessels (arrowed). In the basalis layer (b) and the functionalis layer (c and d) some staining was seen in epithelial cells (G) and in the stroma but staining was weaker than that present in the myometrium (M). IP receptor expression was also present in stromal, cells in the basalis and functionalis layers and in endothelial cells in blood vessels (BV) throughout the endometrium (arrowed).

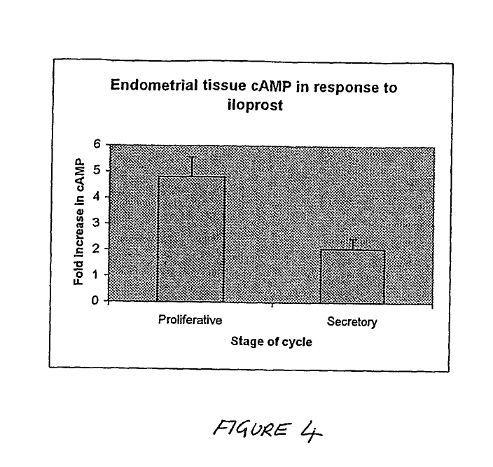

FIG. 4: Cyclic AMP generation in endometrial biopsy tissue in response to 100 nM iloprost. Incubation with iloprost for 10 min in samples obtained from the proliferative phase of the menstrual cycle caused a 4.83±0.74 fold increase in cAMP generation compared with a 2.07±0.38 fold increase from samples obtained from the secretory phase (n=4 for each group; p<0.05).

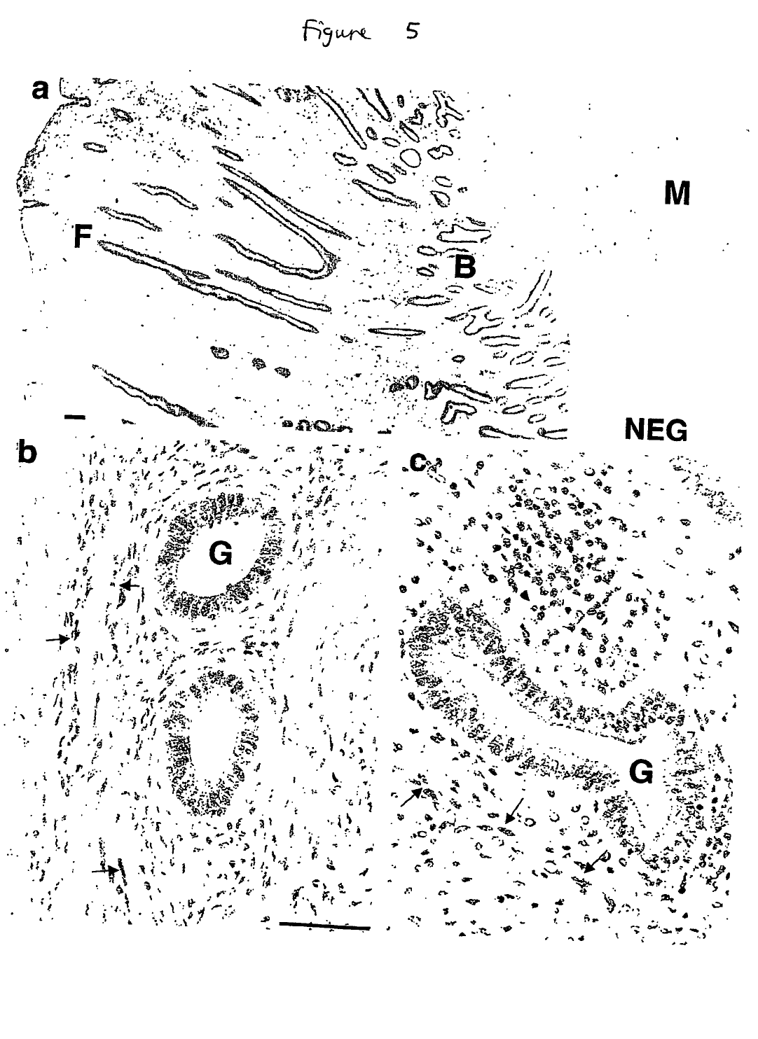

FIG. 5: Immunohistochemical localisation of IP receptor in human endometrial tissue (functional layer and basal-myometrial junction) collected in proliferative phase of the menstrual cycle. Glandular epithelial staining (G) was present in both basal (B) and functional (F) layers and reactivity was detected in smooth muscle cells in the myometrium (M) (FIGS. 5a-c). Stromal cell staining was present throughout the endometrium, but was stronger in the functional layer (FIG. 5c) compared with the basal layer (FIG. 5b). Endothelial cells throughout the endometrium and at basal-myometrial junction also exhibited positive reactivity for IP receptor (indicated by arrows in FIGS. 4b and c). NEG=Negative control; Scale bars=50 μm.

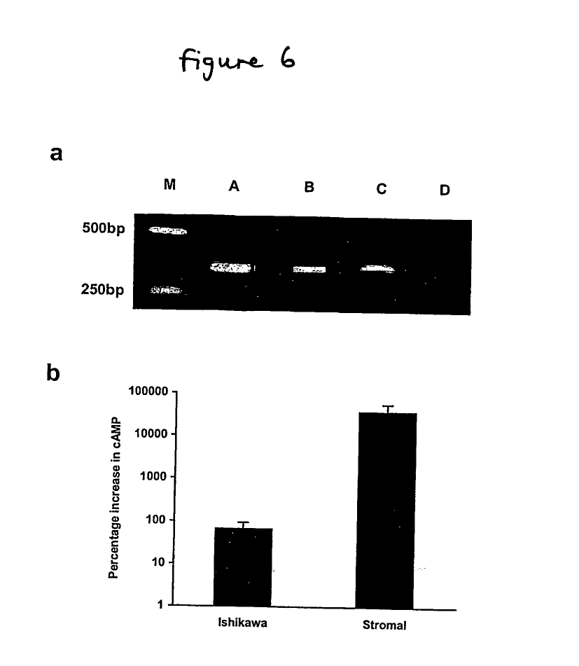

FIG. 6: Expression and signalling of the IP receptor in Ishikawa and ES cells.

FIG. 6a PCR amplification of a 364 bp fragment of human IP receptor in endometrial tissue (Lane A), ES cells (Lane B) and Ishikawa cells (Lane C). Lane D is a water blank and M is DNA marker.

FIG. 6b Cyclic AMP generation in Ishikawa and ES cells in response to 100 nM iloprost (n=3). Results are expressed as the mean±SEM of percentage increase in cAMP induction.

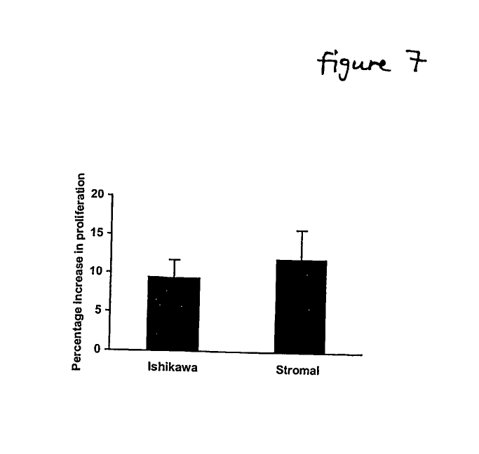

FIG. 7: Proliferation in Ishikawa (n=5) and ES cells (n=6) in response to treatment with 100 nM iloprost for 24 hours. Results are expressed as the mean ±SEM percentage increase in proliferation.

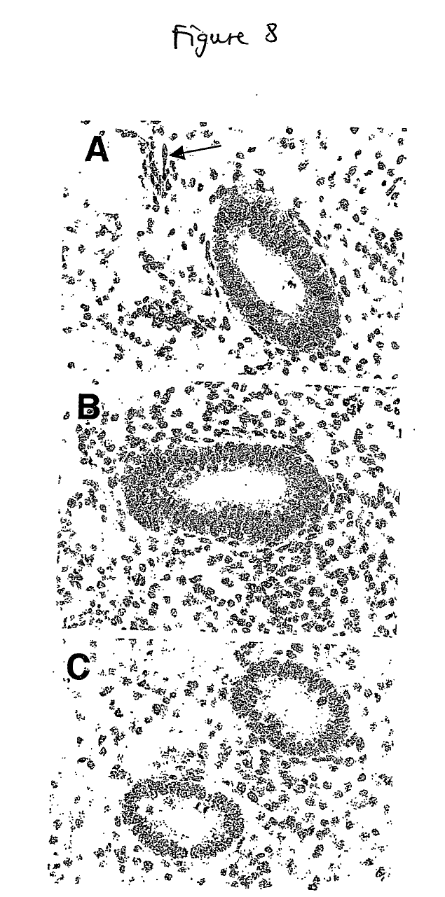

FIG. 8: Immunohistochemistry of IP receptor expression in human endometrium collected from women with excessive (A) or normal (B) blood loss. Section C is a negative control which is tissue subjected to staining with DAB. The arrow indicates a blood vessel.

### EXAMPLE 1

### Temporal Expression, Localisation and Signalling of Prostacyclin (IP) Receptor in the Human Endometrium Across the Menstrual Cycle

Abstract

We have studied the site of expression of prostaglandin I synthase (PGIS) and the prostacyclin receptor (IP) in the non-pregnant human uterus across the menstrual cycle. Using quantitative RT-PCR we demonstrated significantly increased expression of PGIS (P<0.05) in the early proliferative compared with the late proliferative phase. In addition, IP expression was significantly higher in the early proliferative phase of the menstrual cycle compared with the late proliferative and early and late secretory phases (p<0.01 for all phases). Furthermore, in full thickness human uterine biopsies, PGIS was localised by immunohistochemistry to glandular epithelium in basalis and functionalis layers, together with stromal cells in both layers. Staining was also seen in endothelial cells, smooth muscle cells and vascular smooth muscle in the myometrium. IP receptor mRNA was detected by in situ hybridisation, predominantly in proliferative uterus, in myometrium, blood vessels and endometrial epithelial and stromal cells. The prostacyclin analogue iloprost caused cAMP generation in endometrial tissue, which was significantly increased in samples taken from the proliferative phase of the menstrual cycle compared with the secretory phase (p<0.05). We have demonstrated up-regulation of PGIS and IP in the early proliferative phase of the menstrual cycle, with increased signalling of the receptor via the cAMP pathway during this phase

Introduction

Prostacyclin (PGI₂) has been characterised as a vasodilator and a potent inhibitor of platelet aggregation (Smyth & Fitzgerald, 2002) and as such plays an essential role in the maintenance of vascular haemostasis. It is the main prostanoid synthesised by vascular endothelium and acts as a smooth muscle relaxant. Like other members of the eicosanoid family of lipid mediators cyclcooxygenase (COX) is the key enzyme in the synthesis of PGI₂ from arachidonic acid via PGH₂ the common intermediate in prostaglandin synthesis (Kniss, 1999). There are two major isoforms of COX enzymes, COX-1, which is constitutively expressed in many cell types and COX-2, which is induced by many factors including cytokines and tumour promoters. The specific prostaglandin synthase for PGI₂, PGI synthase (PGIS), generates PGI₂ from PGH₂. PGI₂ elicits its effects on target cells by interaction with its G protein-coupled receptor (IP), which has a typical seven-transmembrane structure (Narumiya et al, 1999). The IP receptor can stimulate both Gs and Gq species of G proteins causing an increase in cAMP generation and in phosphatidylinositol response. PGI₂ has been implicated in the aetiology of menorrhagia, which is a major problem in women's reproductive health, accounting for 11% of all gynaecological referrals and incurring drug costs amounting to £7,000,000 in 1995 (Cooper et al, 2001). There is evidence for increased synthesis of PGI₂ (Smith et al, 1981) or an increase in PGI₂ concentration relative to thromboxane A₂ (Makarainen & Ylikorkala, 1986) in women with menorrhagia compared with controls. However, little information is available on the temporal or spatial expression of PGIS or IP in the endometrium across the menstrual cycle.

In the present study, we have studied the site of localisation of PGIS and IP in non-pregnant endometrium and myometrium by quantitative reverse transcriptase polymerase chain reaction (RT-PCR), in situ hybridisation and immunohistochemistry. In addition, we investigated the role of PGI₂ in endometrial cell signalling function by studying the effect of the prostacyclin analogue iloprost on cAMP generation in endometrial tissue samples.

Materials and Methods

Patients and Tissue Collection

Endometrial biopsies at different stages of the menstrual cycle were obtained from women with regular menstrual cycles (25-35 days), who had not received a hormonal preparation in the three months preceding biopsy collection. Samples were collected either with an endometrial suction curette (Pipelle, Laboratoire CCD, France) or as full thickness endometrial biopsies from women undergoing hysterectomy for benign gynaecological indications. Shortly after collection, tissue was either snap frozen in dry ice and stored at −70° C. (for RNA extraction), fixed in neutral buffered formalin (NBF) and wax embedded (for immunohistochemical analyses), or placed in RPMI 1640 (containing 2 mM L-glutamine, 100 U penicillin and 100 μg/ml streptomycin) and transported to the laboratory for in vitro culture.

Biopsies were dated according to stated last menstrual period (LMP) and confirmed by histological assessment according to criteria of Noyes and co-workers (Noyes et al, 1975). Furthermore, circulating oestradiol and progesterone concentrations at the time of biopsy were consistent for both stated LMP and histological assignment of menstrual cycle stage. Ethical approval was obtained from Lothian Research Ethics Committee and written informed consent was obtained from all subjects before tissue collection.

Taqman Quantitative RT-PCR

RNA was extracted from endometrial biopsies obtained from across the menstrual cycle (n=35) using Tri-Reagent (Sigma, Poole, UK) following the Manufacturer's instructions. RNA samples were quantified and were reverse transcribed using 5.5 mM MgCl₂, 0.5 mM each deoxy (d)-NTPs, 2.5 μM random hexamers, ribonuclease inhibitor (0.4 U/μl) and 1.25 U/μl Multiscribe reverse transcriptase (all from Applied Biosystems, Warrington, UK). RNA (400 ng) was added to each reverse transcription reaction and samples were incubated for 90 min. at 25° C., 45 min. at 48° C. and 5 min. at 95° C. The reaction mix for the polymerase chain reaction (PCR) consisted of 1× mastermix, ribosomal 18S forward and reverse primers, ribosomal 18S probe (50 nM; all from ABI), forward and reverse primers for PGIS or IP (300 nM) and PGIS or IP probe (200 nM) (all from Biosource UK Ltd). The reaction mix (48 μl) was aliquoted into tubes and 2 μl cDNA was added. Duplicate 24 μl samples plus positive and negative controls were placed in a PCR plate and wells were sealed with optical caps. The PCR reactions were carried out using an ABI Prism 7700 (Applied Biosystems). All primers and probe were designed using the PRIMER express program (ABI).

The sequences of PGIS primers and probe were: forward, 5′-ACGCAGATGTGGAGATCCCT-3′ (SEQ ID No 27); reverse, 5′-GTCGTGTTCCGGCTGCA-3′ (SEQ ED No 28); and probe (6-carboxy fluoroscein labelled) 5′-CCTCAGCAGGTACGGCTTCGGTCTG-3′ (SEQ ID No 29).

The sequences of the IP primers and probe were: forward, 5′-GCCCTCCCCCTCTACCAA-3′ (SEQ ID No 30); reverse, 5′-TTTTCCAATAACTGTGGTTTTTGTG-3′ (SEQ ID No 31); and probe (6-carboxy fluoroscein labelled) 5′-CCAAGAGCCAGCCCCCTTTCTGC-3′ (SEQ ID No 32).

The sequences of 18S primers and probe have been described previously (Milne et al, 2001).

Data were analysed and processed using Sequence Detector version 1.6.3 (ABI) according to manufacturer's instructions. Results were expressed relative to an internal positive standard included in all reactions.

Non-Quantitative RT-PCR

RNA (1-2 μg per sample) was reverse transcribed using 4 U per reaction of Omniscript reverse transcriptase (Qiagen, Crawley, UK) in 1× reaction buffer containing 0.5 mM of each DNTP, 50 ng/μl Oligo-dT primer and 10 ng/μl random hexamers in a volume of 20 μl. Reverse transcription was for 1 hour at 37° C. followed by 2 minutes at 93° C. A 364 bp fragment of human IP receptor mRNA from base 672 to 1035 was amplified using primers forward, 5′-CAACGGCTCGGTCACCCTCAGC-3′ (SEQ ID No 33) and reverse, 5′-AAGGGGTGTCTGCGAGTCTCCG-3′ (SEQ ID No 34) and HotStarTaq DNA polymerase (Qiagen, Crawley, UK) according to manufacturer's instructions. Five microlitres of each cDNA was added to 2.5 U enzyme, 200 μM of each dNTP and 100 ng of each primer in 50 ml of 1× reaction buffer. Amplification consisted of 1 cycle of 95° C. for 15 minutes, 40 cycles of denaturation at 95° C. for 1 minute, annealing at 66° C. for 30 seconds and extension at 72° C. for 45 seconds. This was followed by a final extension at 72° C. for 10 minutes. Reactions were resolved on a 1% agarose gel and visualised by ethidium bromide transillumination. Sequences of representative pcr products were confirmed by custom sequencing (MWG-Biotech AG, Ebersberg, Germany).

In Situ Hybridisation

A custom synthesised oligonucleotide double fluoroscein isothiocyanate (FITC)-labelled cDNA probe for IP was obtained from Biognostik (Gottingen, Germany). Sections (5 μM) from full thickness human uterine biopsies collected across the menstrual cycle (n=12) were cut onto gelatin-coated slides. Sections were dewaxed and rehydrated and then treated with proteinase K (50 μg/ml in 100 mM Tris-HCl pH 7.6, containing 50 mM EDTA) for 15 mins at 37° C. to enhance cDNA probe access. Sections were washed in diethylpyrocarbonate treated water and pre-hybridised for 4 hours at 30° C. with 25 μl of the hybridisation buffer supplied with the probe, which had been previously heated to 95° C. The sections were then hybridised overnight at 30° C. with the cDNA probe at 6 U/ml in hybridisation buffer. Following hybridisation, sections were washed for 2×5 min. in 1× standard saline citrate (SSC) and 2×15 min. in 0.1×SSC at 39° C. The FITC-labelled probe was detected using standard immununohistochemical reagents with an additional amplification step (TSA Biotin System, NEN Life Sciences, UK). Sections were incubated with blocking buffer for 30 min. Conjugated anti-PITC-horseradish peroxidase (Roche, Diagnostics Ltd., Lewes, UK) was added at a dilution of 1 in 200 in blocking buffer and the sections incubated for 60 min. After washing, biotinyl tyramide amplification reagent (1 in 50) was applied to each slide and incubated for 15 min. Streptavidin-horseradish peroxidase (1 in 100) was applied after washing and incubated for 30 min. and probe localisation visualised with 3,3′-diaminobenzidine (DAB) substrate. Control sections were treated with a double FITC-labelled oligonucleotide probe containing the same proportion of cysteine (C) and guanine (G) bases as the IP probe to assess background hybridization. All treatments were carried out at room temperature unless otherwise specified.

Immunohistochemistry

Endometrial sections (5 μm) from across the menstrual cycle (n=12) were dewaxed in xylene and rehydrated using decreasing grades of ethanol. Antigen retrieval was performed by treating sections for 5 min. in a pressure cooker in boiling 0.1% citrate buffer, pH 3.0. After rinsing in PBS, endogenous peroxidase activity was quenched with 10% H₂O₂ in methanol at room temperature. Non-immune swine serum (20% serum in PBS) was applied for 1 hour before overnight incubation at 4° C. with a rabbit anti-bovine PGIS. An avidin-biotin peroxidase detection system was then applied (DAKO Ltd., Cambridge, UK) with DAB as the chromagen. The anti-PGIS antibody was obtained from Alexis Corporation (Nottingham, UK).

An avidin-biotin peroxidase detection system was then applied (DAKO Ltd., Cambridge, UTK) with DAB as the chromagen. The antibody to IP receptor has been described previously (Fortier et al (2001) Prostaglandins Leukot Essent Fatty Acids 65, 79-83). Non-immune rabbit serum and antibody pre-absorbed with IP receptor peptide were used as controls for IP receptor and non-immune rabbit serum was used as control for PGIS. Immunoreactivity was negligible with pre-absorbed antibody and with non-immune rabbit serum there was occasional generalised pale brown cross reactivity over epithelial glands and endothelium.

Whole Tissue Cyclic AMP Assay

Endometrial biopsies from across the menstrual cycle (n=8) were minced finely with scissors and divided into three portions. The tissue was incubated is overnight at 37° C. in a humidified 5% CO₂ incubator in 2 ml RPMI (Sigma, UK) medium containing, 2 mmol” L-glutamine, 100 IU penicillin and 100 μg streptomycin and 3 μg/ml indomethacin (Sigma, Poole, UK). Following overnight incubation, the tissue was incubated in the same medium containing 1-methyl-3-isobutylxanthine (Sigma) at 37° C. for 30 min. It was then treated with control medium or 100 nM iloprost (a gift from Schering Health Care, Burgess Hill, UK) for 10 min. at 37° C. and lysed in 0.1M HCl and frozen until assayed. cAMP concentration was measured by ELISA (Biomol, Affiniti, Exeter, UK) according to the manufacturer's instructions and normalised to protein concentration determined by a modification of the method of Lowry (Bio-Rad, Hemel Hempstead, UK).

Cyclic AMP and Proliferation Assays in Cell Lines

The Ishikawa endometrial epithelial cell line (Ishikawa cells) and endometrial stromal (ES) cells were propagated in DMEM F12 culture medium with glutamax (Invitrogen Ltd., Paisley, UK) supplemented with 10% foetal bovine serum (PAA Laboratories Ltd., Yeovil, UK). Endometrial stromal cells were a generous gift from Professor R W Kelly and they were derived from human endometrial biopsy tissue as described previously (Dunn et al (2002) J Clin Endocrinol Metab 87, 1898-1901). For cyclic AMP assay, cells were grown in 6 well plates to 70% confluence. They were starved overnight in serum free medium in the presence of indomethacin (3 μg/ml). Cells were then incubated in the same medium containing 1-methyl-3-isobutylxanthine at 37° C. for 30 minutes and treated with control medium or 100 nM iloprost for 10 minutes at 37° C. Following treatment, they were lysed in 0.1M HCl and frozen until assayed. The cyclic AMP assay was performed as for tissue samples. Data are presented as mean±SEM percentage increase in cAMP after treatment with iloprost, where the increase was calculated relative to the control samples. To assess the effect of iloprost on proliferation, cells were seeded in 96 well plates and grown to 70% confluence. They were starved overnight in serum free medium in the presence of indomethacin (3 μg/ml). Cells were treated with control medium or iloprost at a concentration of 100 nM (6 wells for each treatment; data presented as mean of 5 or 6 experiments) for 24 hours at 37° C. Proliferation was assessed using a colorimetric method, the CellTiter 96 Aqueous One Solution Cell Proliferation Assay (Promega, Southampton, UK) according to manufacturer's instructions. Twenty microlitres of the assay reagent (containing a tetrazolium compound, inner salt and an electron coupling reagent) were added to each well and samples were incubated at 37° C. for 2 hours and plates were read at 492 nm. Proliferation is presented as percentage increase relative to untreated cells and plotted as the mean±SEM.

Statistics

Where appropriate, data were subjected to statistical analysis with ANOVA and Fishers PLSD tests (Statview 4.0; Abacus Concepts Inc., USA) and statistical significance accepted when p<0.05.

Results

The pattern of PGIS mRNA expression in the human endometrium across the menstrual cycle was studied by quantitative RT-PCR. PGIS mRNA was detected in all samples of human endometrium examined (FIG. 1a). Relative expression was higher (p<0.05) in the early proliferative phase of the menstrual cycle (10.36±4.58; n=6) than in the late proliferative phase (1.74±0.51; n=7;). PGIS expression was low in the early secretory (3.56+1.89; n=17) or late lo secretory (1.42+0.29; n=4) phases of the menstrual cycle.

The temporal pattern of IP receptor mRNA expression across the menstrual cycle was also assessed by quantitative RT-PCR. IP mRNA was detected in all samples of human endometrium examined by RT-PCR (FIG. 1b). However, the relative expression was significantly higher in the early proliferative phase of is the menstrual cycle (23.28±12.8; n=6) compared with the late proliferative (2.09±0.57; n=7; p<0.01), early secretory (3.16±0.56;n=17; p<0.01) or late secretory (2.08±0.55; n−5; p<0.01) phases.

Immunohistochemical staining for PGIS was performed in full thickness human uterine biopsies obtained from across the menstrual cycle. Cytoplasmic and nuclear staining were present in glandular epithelial cells in the basalis (FIG. 2b) and functionalis FIG. 2c) layers. Stromal cell reactivity was also present in both layers, together with endothelial cell staining in the microvasculature (arrowed in FIG. 2). Myometrial smooth muscle cells showed both cytoplasmic and nuclear reactivity (Inset to FIG. 2a).

The site of expression of IP mRNA was studied by in situ hybridisation. IP reactivity was detected in uterine samples collected during the proliferative phase of the menstrual cycle and was localised in myometrial smooth muscle cells (FIGS. 3a and b) and in endothelial cells lining vessels in all uterine layers (FIGS. 3a and c). Expression was also present in glandular epithelial cells of the basalis and functionalis layers (FIGS. 3b-d), mainly in the cytoplasm, but also overlying nuclei. Stromal cell expression was present in the basalis and functionalis regions (FIGS. 3b-d).

Immunohistochemical staining for IP receptor protein was detected throughout the menstrual cycle in the cytoplasm and nuclei of glandular epithelial cells in both basal and functional layers (FIGS. 5a-c). Similar to PGIS immunoreactivity, IP receptor stromal cell staining was stronger and more widespread in the functional layer (FIG. 5c) compared with the basal layer (FIG. 5b). IP receptor protein expression was also localised to endothelial cells throughout the microvasculature and was present in myometrial smooth muscle cells.

To investigate signalling via the IP receptor, cAMP generation in response to iloprost treatment was assessed in endometrial biopsy tissue (FIG. 4). In biopsy samples, cAMP generation in response to iloprost was significantly higher in endometrial samples collected during the proliferative phase compared with the secretory phase (4.83±0.74 vs 2.07±0.39; n=4 for each group; p<0.05).

To investigate the potential role of the IP receptor in proliferation of epithelial and stromal cells of the endometrium, Ishikawa and ES cells were assessed for expression and signalling of the IP receptor and then utilised for further functional studies. Amplification and sequencing of a 364 bp fragment of IP receptor by conventional techniques confirmed expression of IP receptor in both cell types (FIG. 6a). Moreover, cAMP generation in Ishikawa and ES cells was significantly elevated following treatment with 100 nM iloprost (166%±27.6% and 37,936%±18,464% of control values respectively; n=3; p<0.05) (FIG. 6b).

Finally, the effect of iloprost on proliferation was assessed in the Ishikawa and ES cells (FIG. 7). Treatment with iloprost resulted in a significant increase in proliferation in both Ishikawa and ES cells (109.4%±2.4% and 112%±3.8% of control values respectively; p<0.05).

Discussion

This study demonstrates the expression of PGIS and IP receptor genes in the human endometrium and shows significant upregulation of both during menstruation. PGIS is the terminal enzyme that leads to synthesis of PGI₂ in target tissue (Kniss, D. A. (1999) J Soc Gynecol Investig 6, 285-292). The higher expression level of PGIS during menstruation supports previous observations reporting temporal pattern of PGI₂ secretion by the human endometrium across the menstrual cycle; PGI₂ concentrations are maximal in uterine venous blood during menstruation (Goodfellow et al (1982) Thromb Haemost 48, 9-12). The elevated expression of IP receptor during menstruation suggests that PGI2 synthesis and IP receptor are temporally regulated to induce their effects on target cells. This is supported by the maximal Iloprost induced cAMP response observed in endometrium collected during the menstrual/proliferative phases. The IP receptor has been shown to couple to Gₛ proteins which are linked to increase in cAMP generation (Narumiya et at (1999) Physiol Rev 79, 1193-1226). Although only the protein kinase A pathway was investigated in this study, it is predicted that the IP receptor may activate other signalling pathways in the human endometrium as has been shown recently for other prostaglandin receptors (Milne et al (2003) J Clin Endocrinol Metab 88, 1825-1832; Jabbour et al (2003) J Soc Gynaecol Invest (Supplement) 10, 75). Such diverse signalling pathways may lead to differential activation of target genes that can promote various phenotypic changes on target cells.

The factors that regulate the expression of the PGIS and IP receptor during menstruation in the human endometrium are not clear. It is likely that this temporal expression is regulated by steroid hormones as has been postulated for other prostanoids and their receptors (Milne et al (2001) J Clin Endocriizol Metab 86, 4453-4459; Milne et al (2003) J Clin Endocrinol Metab 88, 1825-1832). Oestradiol-17β has been shown to stimulate the secretion of PGI₂ in endometrial stromal cells (Levin et al (1992) Fertil Steril 58, 530-536). Whether this is associated with up-regulation in expression of IP receptor is unclear. Expression of the IP receptor may be regulated also by PGI₂ or other prostaglandins. Expression of PGIS and synthesis of PGI₂ is induced by COX-2 (Caughey et al (2001) J Immunol 167, 2831-2838), which is up-regulated during the time of menstruation (Jones et al (1997) Hum Reprod 12, 1300-1306). It is also plausible that local mediators within the endometrium may play a role in regulation of PGI₂ synthesis and/or expression of its receptor. For instance, prostacyclin is a mediator of the protective effects of VEGF on the vasculature (Zachary, I. (2001) Am J Physiol Cell Physiol 280, C1375-1386) and PGI₂ biosynthesis is upregulated by VEGF via ERK mediated cPLA₂ activation and arachidonic acid mobilisation (Zachary & Gliki (2001) Cardiovasc Res 49, 568-581).

PGIS and IP receptor expression have been co-localised to multi-cellular compartments of the human endometrium. These include stromal, glandular epithelial, endothelial and smooth muscle cells. This suggests that prostacyclin acts in an autocrine/paracrine manner within the human endometrium to induce its cellular effects. PGIS and IP receptor immunoreactivities have been demonstrated previously in myocytes, vascular smooth muscle cells and endothelial cells in pregnant and non-pregnant human myometrium (Moonen et al (1986) Br J Obstet Gynaecol 93, 255-259; Chegini & Rao (1988) J Clin Endocrinol Metab 66, 76-87; Giannoulias et al (2002) J Clin Endocrinol Metab 87, 5274-5282). To our knowledge, however this the first report of localisation of PGIS and IP receptor in glandular epithelial and stromal cells within the human endometrium. Previous studies using autoradiography with ³[H] PGI₂ on human uterine tissue failed to demonstrate PGI₂ binding sites in epithelial cells (Chegini & Rao (1988) J Clin Endocrinol Metab 66, 76-87), although in that study binding sites were demonstrated in myometrial smooth muscle. This inconsistency with findings presented herein may reflect differences in the sensitivity of the methods used. Interestingly, stromal expression of PGIS and IP receptor was highest in the functional layer of the endometrium. In pre-menopausal women, the human endometrium undergoes phases of proliferation and apoptosis during successive menstrual cycles. These phases are observed predominantly in the functional layer of the endometrium, which is shed at menstruation before regenerating during the proliferative phase of the subsequent menstrual cycle. Hence this spatio-temporal expression of PGIS and IP receptor may be crucial for, and in keeping with, its predicted role in menstruation.

Baird et al ((1996) Eur J Obstet Gynecol Reprod Biol 70, 15-17), have postulated a role for PGI₂ in menstruation based on its myometrial smooth muscle and vascular relaxation effects and inhibition of platelet aggregation. This would counteract the effects of other prostaglandins such as PGF_{2α}, which causes vasoconstriction and myometrial smooth muscle contraction (Crankshaw & Dyal (1994) Can J Physiol Pharmacol 72, 870-874). It is also likely that PGI₂ is involved in the repair of the vascular bed, since it has protective effects on the endothelium by inhibiting vascular smooth muscle proliferation and enhancing endothelial cell survival (Zachary, I. (2001) Am J Physiol Cell Physiol 280, C1375-1386). The increased expression of PGIS and IP receptor during menstruation is also consistent with a role for PGI₂ in the aetiology of menorrhagia. Evidence for this has been provided previously by studies of dysfunctional menstrual bleeding, which have demonstrated increased synthesis of PGI₂ (Smith et al (1981) Lancet 1, 522-524) or increased synthesis of PGI₂ relative to thromboxane A₂ (Makarainen & Ylikorkala (1986) Br J Obstet Gynaecol 93, 974-978) in uterine tissue from women with excessive blood loss relative to controls. Whether IP receptor expression and signalling are also elevated in endometrium of women with dysfunctional menstruation remains to be established.

The role of prostacyclin in endometrial stromal and epithelial cells is unclear. These cells were shown to express functional IP receptors and treatment of both cell types with Iloprost resulted in modest increases in cell proliferation. Prostacyclin has been shown to have both inhibitory and stimulatory effects on proliferation depending on the cell type (Clapp et al (2002) Am J Respir Cell Mol Biol 26, 194-201; Murphy & Fitzgerald (2001) Faseb J 15, 1667-1669). However, the Iloprost induced proliferation observed in endometrial epithelial and stromal cells are much lower than what is previously reported with other prostaglandins such as PGE₂ (25% increase) (Jabbour & Boddy (2003) J Soc Gynaecol Invest (Supplement) 10, 75 and PGF_{2α} (31% increase) (Milne & Jabbour (2003) J Clin Endocrinol Metab 88, 1825-1832). This difference is reflected in the temporal pattern of expression of the IP receptor compared with the EP and FP receptors. Expression of IP receptor is highest during menstruation whereas expression of receptors for PGE₂ (namely EP4) and PGF_{2α} (FP) are detected maximally during the mid-late proliferative phase (Milne et al (2001) J Clin Endocrinol Metab 88, 4453-4459; Milne & Jabbour (2003) J Clin Endocninol Metab 88, 1825-1832). It is tempting to speculate that expression of IP receptors in stromal and epithelial cells may induce genes that are involved in vascular function of the endometrium during menstruation. Expression of a number of these genes such as VEGF, angiopoietins, bFGF or nitric oxide are expressed in the human glandular and/or stromal cells of the endometrium (Gargett & Rogers (2001) Reproduction 121, 181-186; Hewett et al (2002) Am J Pathol 160, 773-780).

In summary, this study has demonstrated temporal expression of PGIS and IP receptor in the non-pregnant human endometrium across the menstrual cycle. Expression of both genes is highest during the menstrual phase and is localised to multi-cellular compartments within the endometrium and myometrium. The function of prostacyclin in the human endometrium is linked to protein kinase A pathway during menstruation. Moreover, treatment of endometrial stromal and epithelial cells with a prostacyclin analogue induces modest increases in proliferation. Future studies will elucidate the role of prostacyclin in menstruation and the mechanisms of its signalling in the human endometrium.

### EXAMPLE 2

### Expression of IP Receptors in Uterine Tissue of Women With Menorrhagia Compared to Women With No Menorrhagia

Endometrial tissue was collected by biopsy from a woman with a known indication of menorrhagia and and a woman who have normal uterine function during the proliferative phase of the menstrual cycle. The tissue was assessed by immunohistochemistry using the method described in Example 1 using the IP receptor antibody (Fortier et al (2001) Prostaglandins Leukol Essent Fatty Acids 65, 79-83) at a dilution of 1/500.

As shown in FIG. 8, expression of the IP receptor is elevated in endometrial tissue from a woman with a known history of menorrhagia (A) compared to a woman with normal blood loss (B).

There is both vascular and glandular epithelial cell staining in the endometrium. Staining m blood vessels suggests that prostacyclin has a direct effect on the versel function such as increased permeability of the vasculature which may lead to increased blood leakage.

Hence, in women with menorrhagia it should prove beneficial to treat with IP receptor antagonists, in order to block the signalling pathway and ultimately transcription of target genes that may mediate vascular function/dysfunction and excessive bleeding.

### EXAMPLE 3

### Treatment of Uterine Cancer With IP Receptor Antagonist

A patient suffering from uterine cancer is administered 3-(5-phenyl-benzofuran-2-ylmethoxycarbonyl-amino)-isonicotinic acid or 2-[5-(4-fluoro-phenyl)-benzofuran-2-ylmethoxycarbonylamino]-5-methanesulfonylamino-benzoic acid at a dosing quantity and frequency such that the therapeutic level of active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 4

### Treatment of Uterine Cancer With an IP Receptor Antagonist and an EP2 Receptor Antagonist

A patient suffering from uterine cancer is administered 4(4-{3-[4(4,5-Dihydro-1H-imidazol-2-ylamino)-phenyl]-propionyl}-3-3-fluoro-phenyl)-piperazine-1-carboxylic acid ethyl ester or 2-[4-(4-isoproxybenzyl)phenyl]amino-imidazoline and AH6809 at a dosing quantity and frequency such that the therapeutic level of active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 5

### Treatment of Uterine Cancer With an IP Receptor Antagonist and an EP4 Receptor Antagonist.

A patient suffering from uterine cancer is administered (R)-3-(1H-benzoimidazol-2-yl)-2-(biphenyl-4-ylmethoxycarbonylamino)-propionic acid or 3-(5-phenyl-benzofuran-2-ylmethoxycarbonyl-amino)-isonicotinic acid and AH23848B at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 6

### Treatment of Uterine Cancer With an IP Receptor Antagonist and an FP Receptor Antagonist

A patient suffering from uterine cancer is administered 2-[5-(4-fluoro-phenyl)-benzofuran-2-ylmethoxycarbonylamino]-5-methanesulfonylamino-benzoic acid or 4-(4-{3-[4-(4,5-Dihydro-1H-imidazol-2-ylamino)-phenyl]-propionyl}-3-fluoro-phenyl)-piperazine-1-carboxylic acid ethyl ester and AL-3138 or AL-8810 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 7

### Treatment of Uterine Cancer With an IP Receptor Antagonist and a COX-2 Inhibitor

A patient suffering from uterine cancer is administered 2-[4-(4-isoproxybenzyl)phenyl]amino-imidazoline or (R)-3-(1H-benzdimidazol-2-yl)-2-(biphenyl-4-ylmethoxycarbonylamino)-propionic acid and nimesulide at a dosing quantity and frequency such that the therapeutic level of active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 8

### Treatment of Fibroids With an IP Receptor Antagonist

A patient suffering from fibroids is administered 4-(4-{3-[4-(4,5-Dihydro-1H-imidazol-2-ylamino)-phenyl]-propionyl}-3-fluoro-phenyl)-piperazine-1-carboxylic acid ethyl ester or 2-[5-(4-fluoro-phenyl)-benzofuran-2-ylmethoxycarbonylamino]-5-methanesulfonylamino-benzoic acid at a dosing quantity and frequency such that the therapeutic level of active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 9

### Treatment of Fibroids With an IP Receptor Antagonist and an EP2 Receptor Antagonist

A patient suffering from fibroids is administered 3-(5-phenyl-benzofuran-2-ylmethoxycarbonyl-amino)-isonicotinic acid or 2-[4-(4-isoproxybenzyl)phenyl]amino-imidazoline and AH6809 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 10

### Treatment of Fibroids With an IP Receptor Antagonist and an EP4 Receptor Antagonist

A patient suffering from fibroids is administered (R)-3-(1H-benzoimidazol-2-yl)-2-(biphenyl-4-ylmethoxycarbonylamino)-propionic acid or 3-(5-phenyl-benzofuran-2-ylmethoxycarbonyl-amino)-isonicotinic acid and AH22921 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 11

### Treatment of Fibroids With an IP Receptor Antagonist and an FP Receptor Antagonist

A patient suffering from fibroids is administered 2-[5-(4-fluoro-phenyl)-benzofuran-2-ylmethoxycarbonylamino]-5-methanesulfonylamino-benzoic acid or (R)-3-(1H-benzoimidazol-2-yl)-2-(biphenyl-4-ylmethoxycarbonylamino)-propionic acid and AL-3 138 or AL-8810 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 12

### Treatment of Fibroids With an IP Receptor Antagonist and a COX-2 Inhibitor

A patient suffering from fibroids is administered 2-[4-(4-isoproxybenzyl)phenyl]amino-imidazoline or 2-[5-(4-fluoro-phenyl)-benzofuran-2-ylnethoxycarbonylamino]-5-methanesulfonylamino-benzoic acid and nimesulide at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 13

### Treatment of Endometriosis With an IP Receptor Antagonist

A patient suffering from endometriosis is administered 3-(5-phenyl-benzofuran-2-ylmethoxycarbonyl-amino)-isonicotinic acid or 4-(4-(3-[4-(4,5-Dihydro-1H-imidazol-2-ylamino)-phenyl]-propionyl}-3-fluoro-phenyl)-piperazine-1-carboxylic acid ethyl ester at a dosing quantity and frequency such that the therapeutic level of active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 14

### Treatment of Endometriosis With an IP Receptor Antagonist and an EP2 Receptor Antagonist

A patient suffering from endometriosis is administered 4-(4-{3-[4-(4,5-Dihydro-1H-imidazol-2-ylamino)-phenyl]-propionyl}-3-fluoro-phenyl)-piperazine-1-carboxylic acid ethyl ester or (R)-3-(1H-benzoimidazol-2-yl)-2-(biphenyl-4-ylmethoxycarbonylamino)-propionic acid and AH6809 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 15

### Treatment of Endometriosis With an IP Receptor Antagonist and an EP4 Receptor Antagonist

A patient suffering from endometriosis is administered 2-[4-(4-isoproxybenzyl)phenyl]amino-imidazoline or 3-(5-phenyl-benzofuran-2-ylmethoxycarbonyl-amino)-isonicotinic acid and AH2291 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 16

### Treatment of Endometriosis With an IP Receptor Antagonist and an FP Receptor Antagonist

A patient suffering from endometriosis is administered 2-[5-(4-fluoro-phenyl)-benzofuran-2-ylmethoxycarbonylamino]-5-methanesulfonylamino-benzoic acid or 2-[4-(4-isoproxybenzyl)phenyl]amino-imidazoline and AL-3138 or AL-8810 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### Example 17

### Treatment of Endometriosis With an IP Receptor Antagonist and a COX-2 Inhibitor.

A patient suffering from endometriosis is administered 4-(4-{3-[4-(4,5-Dihydro-1H-imidazol-2-ylamino)-phenyl]-propionyl}-3-fluoro-phenyl)-piperazine-1-carboxylic acid ethyl ester or (R)-3-(1H-benzoimidazol-2-yl)-2-(biphenyl-4-ylmethoxycarbonylamino)-propionic acid and nimesulide at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 18

### Treatment of Menorrhagia With an IP Receptor Antagonist

A patient suffering from endometriosis is administered 3-(5-phenyl-benzofuran-2-ylmethoxycarbonyl-amino)-isonicotinic acid or 2-[5-(4-fluoro-phenyl)-benzofuran-2-ylmethoxycarbonylamino]-5-methanesulfonylamino-benzoic acid at a dosing quantity and frequency such that the therapeutic level of active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 19

### Treatment of Menorrhagia With an IP Receptor Antagonist and an EP2 Receptor Antagonist

A patient suffering from endometriosis is administered 4-(4-{3-[4-(4,5-Dihydro-1H-imidazol-2-ylamino)-phenyl]-propionyl}-3-fluoro-phenyl)-piperazine-1-carboxylic acid ethyl ester or 2-[4-(4-isoproxybenzyl)phenyl]amino-imidazoline and AH6809 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 20

### Treatment of Menorrhagia With an IP Receptor Antagonist and an EP4 Receptor Antagonist

A patient suffering from endometriosis is administered (R)-3-(1H-benzoimidazol-2-yl)-2-(biphenyl-4-ylmethoxycarbonylamino)-propionic acid or 3-(5-phenyl-benzofuran-2-ylmethoxycarbonyl-amino)-isonicotinic acid and AH2291 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 21

### Treatment of Menorrhagia With an IP Receptor Antagonist and an FP Receptor Antagonist

A patient suffering from endometriosis is administered 2-[5-(4-fluoro-phenyl)-benzofuran-2-ylmethoxycarbonylamino]-5-methanesulfonylamino-benzoic acid or 4-(4-{3-[4-(4,5-Dihydro-1H-imidazol-2-ylamino)-phenyl]-propionyl}-3-fluoro-phenyl)-piperazine-1-carboxylic acid ethyl ester and AL-3 138 or AL-8810 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 22

### Treatment of Menorrhagia With an IP Receptor Antagonist and a COX-2 Inhibitor

A patient suffering from endometriosis is administered 2-[4-(4-isoproxybenzyl)phenyl]amino-imidazoline or (R)-3-(1H-benzoimidazol-2-yl)-2-(biphenyl-4-ylmethoxycarbonylamino)-propionic acid and nimesulide at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 23

### Treatment of Dysmenorrhoea With an IP Receptor Antagonist

A patient suffering from endometriosis is administered 3-(5-phenyl-benzofuran-2-ylmethoxycarbonyl-amino)-isonicotinic acid or 2-[5-(4-fluoro-phenyl)-benzofuran-2-ylmethoxycarbonylamino]-5-methanesulfonylamino-benzoic acid at a dosing quantity and frequency such that the therapeutic level of active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 24

### Treatment of Dysmenorrhoea With an IP Receptor Antagonist and an EP2 Receptor Antagonist

A patient suffering from endometriosis is administered 2-[5-(4-fluoro-phenyl)-benzofuran-2-ylmethoxycarbonylamino]-5-methanesulfonylamino-benzoic acid or 4-(4-{3-[4-(4,5-Dihydro-1H-imidazol-2-ylamino)-phenyl]-propionyl}-3-fluoro-phenyl)-piperazine-1-carboxylic acid ethyl ester and AH6809 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 25

### Treatment of Dysmenorrhoea With an IP Receptor Antagonist and an EP4 Receptor Antagonist

A patient suffering from endometriosis is administered 2-[4-(4-isoproxybenzyl)phenyl]amino-imidazoline or (R)-3-(1H-benzoimidazol-2-yl)-2-(biphenyl-4-ylmethoxycarbonylamino)-propionic acid and AH2291 at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### EXAMPLE 26

### Treatment of Dysmenorrhoea With an IP Receptor Antagonist and an FP Receptor Antagonist

A patient suffering from endometriosis is administered 3-(5-phenyl-benzofuran-2-ylmethoxycarbonyl-amino)-isonicotinic acid or 2-[5-(4-fluoro-phenyl)-benzofuran-2-ylmethoxycarbonylamino]-5-methanesulfonylamino-benzoic acid or 4-(4-{3-[4(4,5-Dihydro-1H-imidazol-2-ylamino)-phenyl]-propionyl}-3-fluoro-phenyl)-piperazine-1-carboxylic acid ethyl ester and AL-3138 or AL-8810 at a dosing quantity and frequency such that the therapeutic level of each active agent at the site of treatment is maintained at a level ideally EC90 but preferably 20 not less than EC50 throughout the treatment period. The treatment is delivered orally or more locally depending on patient acceptability, avoidance of side effects and systemic bioavailability.

### EXAMPLE 27

### Treatment of Dysmenorrhoea With an IP Receptor Antagonist and a COX-2 Inhibitor

A patient suffering from endometriosis is administered 2-[4-(4-isoproxybenzyl)phenyl]amino-imidazoline or (R)-3-(1H-benzoimidazol-2-yl)-2-(biphenyl-4-ylmethoxycarbonylamino)-propionic acid and nimesulide at a dosing quantity and frequency such that the therapeutic level of each active agent at the site of treatment is maintained at a level ideally EC90 but preferably not less than EC50 throughout the treatment period. The treatment is delivered orally or more locally depending on patient acceptability, avoidance of side effects and systemic bioavailability.

### REFERENCES

Smyth E M, FitzGerald G A (2002) Human prostacyclin receptor. Vitam Horm 65: 149-165.
Kniss D A (1999) Cyclooxygenases in reproductive medicine and biology. J Soc Gynecol Investig 6: 285-292.
Narwniya S, Sugimoto Y, Ushikubi F (1999) Prostanoid receptors: structures, properties, and functions. Physiol Rev 79: 1193-1226.
Cooper K G, Jack S A, Parkin D E, Grant A M (2001) Five-year follow up of women randomised to medical management or transcervical resection of the endometrium for heavy menstrual loss: clinical and quality of life outcomes. Bjog 108: 1222-1228.
Smith S K, Abel M H, Kelly R W, Baird D T (1981) A role for prostacyclin (PGi2) in excessive menstrual bleeding. Lancet 1: 522-524.
Makarainen L, Ylikorkala O 1986 Primary and myoma-associated menorrhagia: role of prostaglandins and effects of ibuprofen. Br J Obstet Gynaecol 93: 974-978.
Noyes R W, Hertig A T, Rock J 1975 Dating the endometrial biopsy. Am J Obstet Gynecol 122: 262-263.
Milne S A, Perchick G B, Boddy S C, Jabbour H N (2001) Expression, localization, and signaling of PGE(2) and EP2/EP4 receptors in human nonpregnant endometrium across the menstrual cycle. J Clin Endocrinol Metab 86: 4453-4459.
Ashby, B. (1998) Co-expression of prostaglandin receptors with opposite effects: a model for homeostatic control of autocrine and paracrine signalling. Biochem Pharmacol 55: 239-246.
Coleman, R A, Smiith, W L, Narumiya, S. (1994) International Union of Phannacology classification of prostanoid receptors: properties, distribution, and structure of the receptors and their subtypes. Pharmacol Rev 46: 205-229.
DeWitt, D L. (1991) Prostaglandin endoperoxide synthase: regulation of enzyme expression. Biochim Biophys Acta 1083: 121-134.
Gordon, M D, Ireland, K. (1994) Pathology of hyperplasia and carcinoma of the endometrium. Semin Oncol 21: 64-70.
Herschman, H R. (1996) Prostaglandin synthase 2. Biochlim Biophys Acta 1299: 125-140.
Mant, J W, Vessey, M P. (1994) Ovarian and endometrial cancers. Cancer Surv 20: 287-307.
Subbaramaiah, K, Telang, N, Ramonetti, J T, Araki, R, DeVito, B, Weksler, B B, Dannenberg, A J. (1996) Transcription of cyclooxygenase-2 is enhanced in transformed mammary epithelial cells. Cancer Res 56: 4424-4429.

1. A method of preventing or treating a pathological condition of the uterus in a female individual, the method comprising administering to the individual at least one agent that is an antagonist of the IP receptor and/or an inhibitor of prostaglandin I synthase (PGIS).

2. The method according to claim 1 wherein the pathological condition of the uterus is associated with abnormal growth of cells of the myometrium or endometrium.

3. The method according to claim 1 wherein the pathological condition of the uterus is selected from uterine carcinoma, menorrhagia, dysmenorrhoea and an endometrial or myometrial pathological condition.

4. The method according to claim 3 wherein the endometrial pathological condition is endometriosis.

5. The method according to claim 3 wherein the myometrial pathological condition is fibroids.

6. The method according to any of claim 1 wherein the antagonist of the IP receptor prevents or reduces the binding of PGI2 to the IP receptor.

7. The method according to any of claim 1 wherein the antagonist of the IP receptor affects the interaction between PGI2 and the IP receptor, or the interaction between the IP receptor and the associated G protein, thus inhibiting or disrupting a PGI2-IP mediated signal transduction pathway.

8. The method according to claim 1 wherein the IP receptor antagonist is any one or more of a 2-(arylphenyl)amino-imidazoline derivative a 2-(substituted-phenyl)amino-imidazoline derivative an alkoxycarbonylamino heteroaryl carboxylic acid derivative an alkoxycarbonylamino benzoic acid or alkoxycarbonylamino tetrazolyl phenyl derivative a 2-phenylaminoimidazoline phenyl ketone derivative a carboxylic acid derivative an amino- or amido-prostacyclin derivative compound a 15(R)-isocarbacyclin or 15-deoxyisocarbacyclin derivative a 6,9-thiaprostacyclin analogue or derivative (5Z)-carbacyclin; FCE 22176 ((5Z)-13,14-didehydro-20-methyl-carboprostacyclin); and an anti-IP receptor antibody.

9. The method according to claim 1 wherein the agent is an antagonist of PGI2.

10. The method according to claim 1 wherein the agent is an inhibitor of PGIS.

11. The method according to claim 10 wherein the PGIS inhibitor is an anti-PGIS antibody, U-51605, peryoxynitrite, 3-morpholinosydnonimine N-ethylcarbamide or trans-2-phenylcyclopropylamine HCl.

12. The method according to claim 1 further comprising administering to the individual an inhibitor of PGES and/or an antagonist of EP2 or EP4.

13. The method according to claim 12 wherein the antagonist of EP2 or EP4 is one or more of AH6809, an omega-substituted prostaglandin E derivative AH23848B, AH22921X, IFTSYLECL, IFASYECL, IFTSAECL, IFTSYEAL, ILASYECL, IFTSTDCL, TSYEAL (with 4-biphenylalanine), TSYEAL (with homophenylalanine), a 5-thia-prostaglandin E derivative 5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt, 5-butyl-2,4-dihydro-4-[[2′-[N-(2-methyl-3-furoyl)sulfamoyl]biphenyl-4-yl]methyl]-2-{2-(trifluoromethyl)phenyl]-1,2,4-triazol-3-one, 5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one, 5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one, and 5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.

14. The method according to claim 1 further comprising administering to the individual an agent that is an antagonist of the FP receptor.

15. The method according to claim 14 wherein the FP receptor antagonist is any one or more of PGF2α dimethyl amide; PGF2α dimethyl amine; AL-8810 ((5Z,13E)-(9S,11S,15R)-9,15-dihydroxy-11-fluoro-15-(2-indanyl)-16,17,18,19,20-pentanor-5,13-prostadienoic acid); AL-3138 (11-deoxy-16-fluoro PGF2α); phloretin; glibenclamide; ridogrel; PHG113; PCP-1 (rvkfksqqhrqgrshhlem); PCP-2 (rkavlknlyklasqccgvhvislhiwelssiknslkvaaisespvaeksast); PCP-3 (clseeakearrindeierqlrrdkrdarre-NH2); PCP-4 (kdtilqlnlkeynlv-NH2); PCP-8 (ilghrdyk); PCP-10 (wedrfyll); PCP-13 (ILGHRDYK); PCP-14 (YQDRFYLL); (ILAHRDYK); PCP-13.7 (ILAHRDYK); PCP-13.8 (ILaHRDYK); PCP-13.11 (ILGFRDYK); PCP-13.13 (ILGHKDYK); PCP-13.14 (ILGHRNYK); PCP-13.18 (ILGHQDYK); PCP-13.20 (ILGHRDY-amide); PCP-13.21 (ILGHRDYK-amide); PCP-13.22 (ILGWRDYK); PCP-13.24 (ILGXRDYK); and PCP-15 (SNVLCSIF).

16. The method according to claim 14 wherein the agent that is an antagonist of the FP receptor is an antagonist of PGF2α.

17. The method according to claim 16 wherein the PGF2α antagonist is an anti-PGF2α antibody.

18. The method according to claim 1 further comprising administering to the individual a cyclooxygenase-2 (COX-2) inhibitor.

19. The method according to claim 18 wherein the COX-2 inhibitor is any one of nimesulide, 4-hydroxynimesulide, flosulide, and meloxicam.

20-42. (canceled)

43. A composition comprising at least one agent that is an antagonist of the IP receptor and/or a PGIS inhibitor, and any one or more of an inhibitor of PGES and/or an antagonist of EP2 or EP4, an agent that is an antagonist of the FP receptor, and a COX-2 inhibitor.

44. (canceled)

45. A pharmaceutical composition comprising the composition according to claim 43 and a pharmaceutically acceptable carrier.

46. A vaginal ring or a tampon or an intrauterine device comprising the composition according to claim 43.

47-50. (canceled)

51. The method according to claim 1 further comprising administering to the individual any one or more of an inhibitor of PGES and/or an antagonist of EP2 or EP4, an agent that is an antagonist of the FP receptor, and a COX-2 inhibitor.

52. The method according to claim 12 further comprising administering to the individual an agent that is an antagonist of the FP receptor.

53. The method according to claim 12 further comprising administering to the individual a COX-2 inhibitor.

54. The method according to claim 14 further comprising administering to the individual a COX-2 inhibitor.

55. The method according to claim 1, wherein the at least one agent is administered via a vaginal ring or a tampon or an intrauterine device.

56. The composition according to claim 43, wherein the antagonist of the IP receptor prevents or reduces the binding of PGI2 to the IP receptor.

57. The composition according to claim 43, wherein the antagonist of the IP receptor affects the interaction between PGI2 and the IP receptor, or the interaction between the IP receptor and the associated G protein, thus inhibiting or disrupting a PGI2-IP mediated signal transduction pathway.

58. The composition according to claim 43, wherein the IP receptor antagonist is any one or more of a 2-(arylphenyl)amino-imidazoline derivative; a 2-(substituted-phenyl)amino-imidazoline derivative; an alkoxycarbonylamino heteroaryl carboxylic acid derivative; an alkoxycarbonylamino benzoic acid or alkoxycarbonylamino tetrazolyl phenyl derivative; a 2-phenylaminoimidazoline phenyl ketone derivative; a carboxylic acid derivative; an amino- or amido-prostacyclin derivative compound; a 15(R)-isocarbacyclin or 15-deoxyisocarbacyclin derivative; a 6,9-thiaprostacyclin analogue or derivative; (5Z)-carbacyclin; FCE 22176 ((5Z)-13,14-didehydro-20-methyl-carboprostacyclin); and an anti-IP receptor antibody.

59. The composition according to claim 43, wherein the agent is an antagonist of PGI2.

60. The composition according to claim 43, wherein the agent is an inhibitor of PGIS.

61. The composition according to claim 60, wherein the PGIS inhibitor is an anti-PGIS antibody, U-51605, peryoxynitrite, 3-morpholinosydnonimine N-ethylcarbamide or trans-2-phenylcyclopropylamine HCl.

62. The composition according to claim 43, wherein the antagonist of EP2 or EP4 is one or more of AH6809, an omega-substituted prostaglandin E derivative, AH23848B, AH22921X, IFTSYLECL, IFASYECL, IFTSAECL, IFTSYEAL, ILASYECL, IFTSTDCL, TSYEAL (with 4-biphenylalanine), TSYEAL (with homophenylalanine), a 5-thia-prostaglandin E derivative, 5-butyl-2,4-dihydro-4-[[2′-[N-(3-chloro-2-thiophenecarbonyl)sulfamoyl]biphenyl-4-yl]methyl]-2-{2-(trifluoromethyl)phenyl]-1,2,4-triazol-3-one potassium salt, 5-butyl-2,4-dihydro-4-[[2′-[N-(2-methyl-3-furoyl)sulfamoyl]biphenyl-4-yl]methyl]-2-{2-(trifluoromethyl)phenyl]-1,2,4-triazol-3-one, 5-butyl-2,4-dihydro-4-[[2′-[N-(3-methyl-2-thiophenecarbonyl)sulfamoyl]biphenyl-4-yl]methyl]-2-{2-(trifluoromethyl)phenyl]-1,2,4-triazol-3-one, 5-butyl-2,4-dihydro-4-[[2′-[N-(2-thiophenecarbonyl)sulfamoyl]biphenyl-4-yl]methyl]-2-{2-(trifluoromethyl)phenyl]-1,2,4-triazol-3-one, and 5-butyl-2,4-dihydro-4-[[2′-[N-[2-(methypyrrole)carbonyl]sulfamoyl]biphenyl-4-yl]methyl]-2-{2-(trifluoromethyl)phenyl]-1,2,4-triazol-3-one.

63. The composition according to claim 43, wherein the FP receptor antagonist is any one or more of PGF2a dimethyl amide; PGF2α dimethyl amine; AL-8810 ((5Z,13E)-(9S,11 S,15R)-9,15-dihydroxy-11-fluoro-15-(2-indanyl)-16,17,18,19,20-pentanor-5,13-prostadienoic acid); AL-3138 (11-deoxy-16-fluoro PGF2α); phloretin; glibenclainide; ridogrel; PHG113; PCP-1 (rvkfksqqhrqgrshhlem); PCP-2 (rkavlknlyklasqccgvhvislhiwelssiknslkvaaisespvaeksast); PCP-3 (clseeakearrindeierqlrrdkrdarre-NH2); PCP-4 (kdtilqlnlkeynlv-NH2); PCP-8 (ilghrdyk); PCP-10 (wedrfyll); PCP-13 (ILGHRDYK); PCP-14 (YQDRFYLL); (ILAHRDYK); PCP-13.7 (ILAHRDYK); PCP-13.8 (ILaHRDYK); PCP-13.11 (ILGFRDYK); PCP-13.13 (ILGHKDYK); PCP-13.14 (ILGHRNYK); PCP-13.18 (ILGHQDYK); PCP-13.20 (ILGHRDY-amide); PCP-13.21 (ILGHRDYK-amide); PCP-13.22 (ILGWRDYK); PCP-13.24 (ILGXRDYK); and PCP-15 (SNVLCSIF).

64. The composition according to claim 43, wherein the agent that is an antagonist of the FP receptor is an antagonist of PGF2α.

65. The composition according to claim 64, wherein the PGF2α antagonist is an anti-PGF2α antibody.

66. The composition according to claim 43, wherein the COX-2 inhibitor is any one of nimesulide, 4-hydroxynimesulide, flosulide, and meloxicam.
